(19) United States
(12) Patent Application Publication
(10) Pub. No.: US 2020/0345979 A1
(43) Pub. Date: Nov. 5, 2020

(54) Transradial Access Systems Particularly Useful for Cerebral Access

(71) Applicants: Yince Loh, Bainbridge Island, WA (US); Akshal Patel, Seattle, WA (US)

(72) Inventors: Yince Loh, Bainbridge, WA (US); Akshal Patel, Seattle, WA (US)

(21) Appl. No.: 16/827,507

(22) Filed: Mar. 23, 2020

Related U.S. Application Data
(60) Provisional application No. 62/822,538, filed on Mar. 22, 2019.

(51) Int. Cl. A61M 25/00; A61M 25/10

(52) U.S. Cl. CPC A61M 25/0041; A61M 25/0068; A61M 25/104; A61M 2210/0693; A61M 2025/0057; A61M 2210/125; A61M 25/0013

(57) ABSTRACT

Transradial access of mammalian vasculature enhances and renders novel approaches to the heart, brain and other associated organ systems less fraught with risk and more appropriate for ageing populations and challenging vessel morphologies, inter alia. Cerebral access is emphasized.

### CROSS REFERENCE TO RELATED APPLICATIONS

This application claims priority benefit from U.S. Provisional Patent Application Ser. No. 62/822,538, filed Mar. 22, 2019, the content of which is incorporated herein by reference in its entirety.

### FIELD OF THE DISCLOSURE

The disclosure relates to systems, medical devices, catheters, and methods for diagnosing or treating disorders of the aortic arch, carotid artery, and disorders of associated arteries including supra-aortic vessels. Treating disorders encompasses placement of medical devices such as stents or angioplasty balloons, and encompasses removal of atherosclerotic plaque by abatement or by enzymatic methods.

### BACKGROUND OF THE DISCLOSURE

The present disclosure provides a catheter and related methods for transradial access to the heart, brain, and associated blood vessels and associated organs. The catheter and related methods overcome difficulties in placing a medical devices into the carotid artery by allowing ease of access to this artery. Treatment of disorders of aortic arch and treatment of disorders of supra-aortic vessels encompasses, for example, inserting a stent, inserting and operating an angioplasty balloon, administering one or more drugs, physical methods for removing atherosclerotic plaque, and physical methods for removing a blood clot or an embolism.

Physical methods for removing plaque includes, atherectomy for debulking the plaque burden within a blood vessel. Debulking by atherectomy catheter can use a balloon opposite a cutting blade for maximizing plaque removal (see, Franzone, Piccolo, Trimarco (2012) BMC Surgery. 12 (Suppl. 1):S13). Catheter can be used for aspirating material from plaque or from embolism. What can be removed is hard plaque, soft plaque, calcium deposits, thrombus, adhering platelets, and fibrotic lesions. Catheter of the present disclosure can be used to insert a filter in a blood vessel that is downstream of the plaque, in order to prevent dispersion of lesional material throughout the circulatory system. Also, catheter of the present disclosure can be used to deliver enzymes for enzyme-catalyzed debulking of atherosclerotic plaque from a blood vessel, such as carotid artery, with or without using traumatic methods for debulking plaque (see, Wang, Mansukhani (2019) J. Surgical Res. 233:335-344). Also, catheter of the present disclosure can be used for providing ultraviolet light or laser light (laser atherectomy) for debulking atherosclerotic plaque (see, Bhat, Afari, Garcia (2017) J. Invasive Cardiol. 29:135-144).

### BRIEF DESCRIPTIONS OF THE DRAWINGS

How to describe shapes of catheter, including shapes of regions or segments of catheter. For describing non-alternative embodiments of catheter and alternative embodiments of catheter, the following terminology is used. For description purposes, the term “segment” is used to refer to various regions of catheter. The term “segment” does not imply that each of these regions are manufactured separately and then attached together, and it does not imply that each of these regions have different types of coatings. Length of each segment of catheter is measured along the axis of the catheter (measured along the center of the lumen of each segment). Segments can differ from one another by their length, by their external diameter (French size), by curvature, by taper, by their coating, and so on.

Where catheter has two or more curved segments, the relative orientation of the curved segments is established by way of two independent parameters: (First parameter) The positioning of each segment relative to each other when traveling from the end closest to the hub (proximal end) to the end farthest from the hub (distal end); and (Second parameter) The positioning of each curved segment relative to each other, according to whether central point of the first segment is on the same side (or on the opposite side) of the catheter as the central point of the second segment.

For any curved segment, the curved segment defines a plane (defines a flat surface where this flat surface has a 2-dimensional area). Catheter embodiments of the present disclosure encompass catheter embodiments where the curve defines a two-dimensional area, and also, encompass catheter embodiments where a curved segment defines a three-dimensional volume.

Relaxed orientation of catheter versus flexed orientation of catheter. Unless specified otherwise, either explicitly or by the context, every description of catheter embodiments of the present disclosure refers to a catheter that assumes a relaxed orientation. Relaxed orientation refers to the orientation, for example, when the physician clutches the catheter by only the hub and where the rest of the catheter does not contact any solid or liquid. Alternatively, relaxed orientation refers to the orientation of catheter, when catheter rests on a table top. But flexed orientation refers to catheter where physician compresses distal end of catheter, for example, where the goal is to straighten out the semicircular segment. Also, flexed orientation refers to catheter when it is situated inside patient's vasculature, and where anatomy of vasculator forces catheter to assume shapes that are different from the relaxed orientation.

Orientation of curved segments, with respect to each other (oriented in same way versus oriented in opposite way). Defining orientation by the “central point.” For describing whether a first curved segment that defines a flat 2-dimensional area, is oriented in substantially the same way or oriented in a generally opposite way than a second curved segment that also defines a flat 2-dimensional area, it is assumed that the curve of each curved segment is associated with a central point (to illustrate this, when drawing a curve using a compass, and where the compass has a shaft of graphite attached to one arm and a sharp metal point on the other arm, the location on the paper where the sharp metal point is anchored is what is this central point). Design patent D246,981 of Yamaguchi shows a compass with a first arm with a clamp for securing a shaft of graphite and a second arm with a sharp metal point.

FIG. 1 shows the young aortic arch (Type I aortic arch). For the purpose of comparing the young aortic arch with variants of aortic arch, the young arch may be considered to be the “normal” form of the aortic arch. Can be accessed by either femoral artery or radial artery.

FIG. 2 shows the Bovine Arch variant (Type II aortic arch). Difficult access by way of femoral artery, but feasible for access with access by way of radial artery.

FIG. 3 shows the Unwound (Aged) Arch variant (Type III aortic arch). Type I arch has “elongation and rostral migration of the arch, with the brachial artery trunk originating lower than the left subclavian artery by more than 2 diameters of the left common carotid artery” (Wagdi (2013) Cardiol. Res. 4:8-14). Difficult access by way of femoral artery, but feasible with access by way of radial artery.

FIG. 4 discloses a guidewire that includes a microwire segment. Guidewire is unique in that the distal part of the guidewire is a microwire, which is typically considered 0.020 inches or smaller, but has a body and proximal shaft that is of a normal wire, which is 0.035 inches to 0.038 inches.

FIG. 5 discloses basic design catheter that has a simpler design than those shown in FIG. 6 and FIG. 7.

FIG. 6 discloses catheter with an S-shaped segment, where this S-shaped segment resides in between semicircular segment and straight tapered segment.

FIG. 7 shows a catheter with flipped-tip segment that resides at the end of the catheter that is farthest from the hub. Flipped-tip segment consists of a first segment and a second segment, where the first segment is a 0.5 centimeter straight segment and a 1.0 centimeter curved segment with a 45 degree arc (arc that assumes a cut-out from a circle)

FIG. 8 shows balloon tip sheath.

FIG. 9A shows top view of board base, spot for wrist pad, attachment section, and fluoro table width.

FIG. 9B shows edge-on view of board base and ledge.

FIG. 10A shows top view of wrist pad, strap holes for grip bar, and width of wrist pad when viewed from the top.

FIG. 10B shows edge-on view of wrist pad, strap holes for wrist bar, and width of wrist pad when viewed from the edge.

FIG. 11 shows grip bar.

FIG. 12A shows top view of table top, support stand, access site, exposure cutaway, and gutter to which drain bag attaches.

FIG. 12B shows side view of table top, lengths on the table top, curving ledge of the table top, and support stand.

FIG. 13. Curved segments that share a common axis, where one or more of the segments can be used to define part of a circle. This figure can be used to define part or all of a curved region of catheter of the present invention. In various embodiments, a curved region of the catheter can include a curved region that is acquired from (carved out of) the image of FIG. 13. This acquired region can represent the entire curved region. Alternatively, this acquired region can be combined with zero, one, or more regions acquired from FIG. 14 (stretched area of oval) and with zero, one, or more regions acquired from FIG. 15 (squashed area of oval). It is possible that most or perhaps all of the curved segments of catheter of the present disclosure can be defined by segments of the semicircle of FIG. 13. For example, curves that appear to be almost flat can be defined as a ten degree slice from the curve of FIG. 13. But using FIG. 13 to define this almost flat curve to guide in manufacturing a catheter is almost impossible, because the user won't be able to compare this ten degree slice with a catheter that needs to be manufactured. To overcome this problem with perceiving, visualizing, and comparing, the curve of FIG. 14 can be used to define curves that are almost flat.

FIG. 14. Curved segments that share a common axis, where one or more of the segments can be used to define part of the stretched-out region of an oval (stretched, with respect to a circle). This figure can be used to define part or all of a curved region of catheter of the present invention. This defined region can be combined with zero, one, or more regions acquired from FIG. 13 and with zero, one, or more regions from FIG. 15.

FIG. 15. Curved segments that share a common axis, where one or more of the segments can be used to define part of the squashed region of an oval (squashed, with respect to a circle). This figure can be used to define part or all of a curved region of catheter of the present invention. This defined region can be combined with zero, one, or more regions from FIG. 13, and with zero, one, or more regions from FIG. 14.

### SUMMARY OF THE DISCLOSURE

In embodiments, what is provided is A catheter that is capable of radial passage through the cardiovascular system, and capable of passage to the lumen of the aortic arch, wherein the catheter comprises a proximal end and a distal end, and wherein the proximal end to the distal end of the catheter comprises a lumen, and wherein the catheter comprises: (i) An optional hub that comprises the proximal end, (ii) A first straight segment, that is not tapered, (iii) A second straight segment that is tapered, (iv) Optionally, a third straight segment, that is not tapered, (v) A third straight segment, that is not tapered, (vi) A first curved segment, (vii) A second curved segment, and (viii) Optionally, an S-shaped region consisting of a first curved segment that resides in this S-shaped region, and a second curved segment that resides in this S-shaped region, wherein the first curved segment provides the bottom half of an S-shape, and the second curved segment provides the top half of the S-shape, wherein the bottom half is relatively proximal and the top half is relatively distal.

### First Embodiment (Basic Design Embodiment)

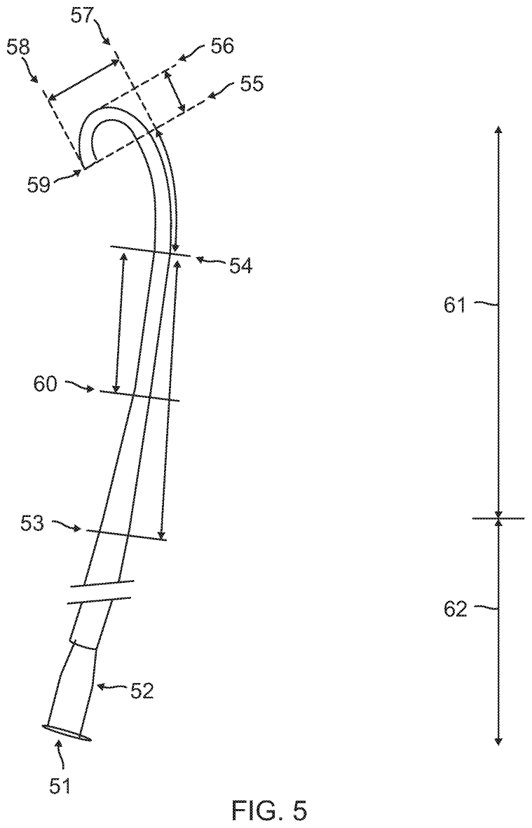

What is provided is the above catheter, wherein the catheter is exemplified by FIG. 5, wherein the catheter comprises a proximal end and a distal end, wherein the proximal end to the distal end the catheter comprises a lumen, wherein the catheter comprises: (i) A hub that comprises the proximal end, (ii) A first straight segment, that is not tapered, (iii) A second straight segment, that is tapered, (iv) A third straight segment, that is not tapered, (v) A first curved segment, and (vi) A second curved semicircular segment that comprises the distal end of said catheter, wherein the first curved segment and the second curved segment assume a continuously curving arc, and wherein the first curved segment possesses a first central point, and the second curved segment possesses a second central point, and wherein the first and second central points are relatively close to each other, wherein the hub defines an axis, and wherein each segment defines an axis, wherein the combined length of the hub plus the first straight segment has a value that is selected from a value that is between 100 cm to 115 cm, wherein the second straight segment that is tapered is about three centimeters long and has a taper that gets narrower from the proximal direction to the distal direction, wherein taper begins at 6 French (outer diameter) and ends at 4 French (outer diameter), wherein the third straight segment has a length of 3 centimeters and a constant width of 4 French (outer diameter), wherein the first curved region has a constant width of 4 French, and a length of about 3 centimeters a measurable along central axis of first curved region, wherein the first curved region assumes a thirty degrees arc, wherein the second curved segment is a semicircular (180 degrees) segment with constant width of 4 French, and wherein second curved segment comprises the distal terminus of the catheter, and wherein the semicircle has an outer circumference diameter of 1.5 cm, and a radius of 0.75 cm, and wherein catheter has a total length measurable along central axis, wherein total catheter length is 125 cm or 125 cm, and wherein the distal tip defines an aperture with 4 French outer diameter and 0.038 inch inner diameter, and wherein the arc of the distal curve and the arc of the semicircle together form an arc that has a J-shaped conformation and does not have an S-shaped conformation.

### Second Embodiment of Catheter (S-Curve Embodiment)

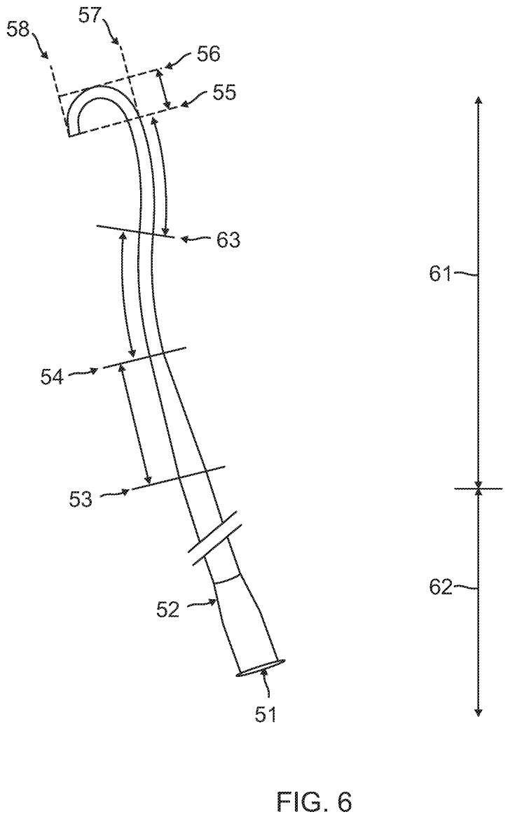

In another embodiment, what is provided is the above catheter, wherein the catheter is exemplified by FIG. 6, wherein the catheter comprises a proximal end and a distal end, wherein the catheter comprises a lumen, and wherein the catheter comprises: (i) A hub that is located at the proximal end, (ii) A first straight segment, that is not tapered, (iii) A second straight segment, that is tapered, (iv) An S-shaped curve that comprises first curved segment and a second curved segment, (v) A third curved segment that comprises an aperture open to the environment of use, wherein the third curved segment also comprises said distal end, wherein the third curved segment terminates in a distal tip that comprises an aperture that opens into environment of use, and wherein the hub defines an axis and wherein each segment defines an axis, and wherein said first curved segment and said second curved segment assume an S-shaped curve, and wherein said second curved segment and said third curved segment assume a continuously curving arc that assumes a continuous J-shaped curve, wherein the combined length of the hub plus the first straight segment has a value that is selected from a value that is between 100 cm to 115 cm, wherein the total catheter length is 115 cm or 125 cm, wherein the straight tapered segment is three centimeters long as measurable by axis of lumen of straight tapered segment, wherein the straight tapered segment has a taper that gets narrower from the proximal direction to the distal direction, wherein taper begins at 6 French (outer diameter) and ends at 4 French (outer diameter), wherein the first curved region has a constant width of 4 French, and is about three centimeters long as measurable along central axis of first curved region, wherein the second curved region has constant width of 4 French and is about three centimeters long, wherein the third curved segment is a semicircular (180 degrees) segment with constant width of 4 French, and wherein the third curved segment comprises the distal terminus of the catheter, and wherein the semicircle has an outer circumference diameter of 1.5 cm and a side-to-side radius of 0.75 cm, and a distal-to-proximal radius of 0.50 cm, and wherein the catheter has a total length measurable along central axis, and wherein the distal tip possesses an aperture with 4 French outer diameter and 0.038 inch inner diameter, and wherein the aperture is open to the environment of use. The S-curved region allows for an easier engagement of the arteries from a radial approach, specifically the right carotid and a bovine left carotid.

### Third Embodiment (Flipped-Tip Embodiment)

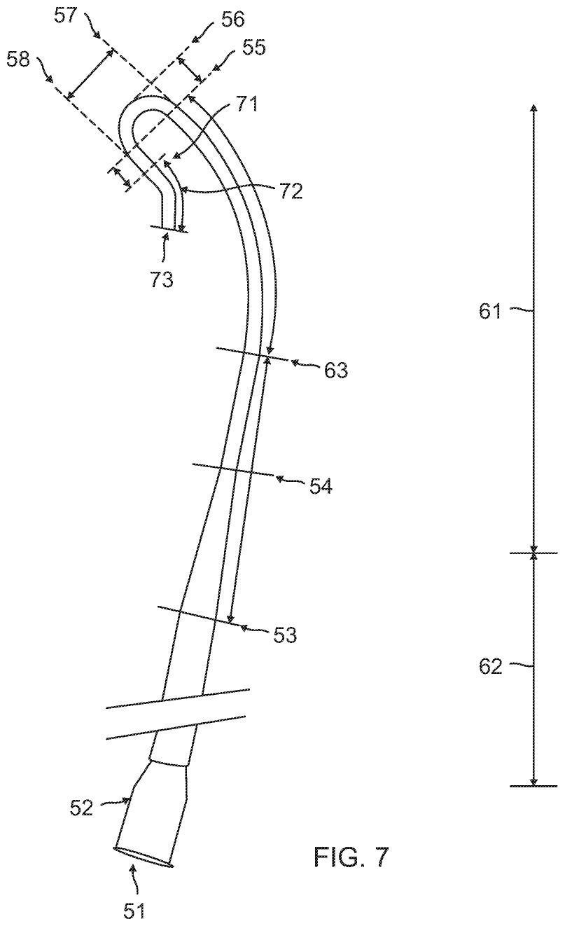

In another aspect, what is provided is the above catheter, wherein the catheter is exemplified by FIG. 7, wherein the catheter comprises a proximal end and a distal end, wherein the catheter defines a lumen that extends from proximal end to distal end, wherein the catheter comprises: (i) A hub that is located at proximal end, (ii) A first straight segment, that is not tapered, (iii) A second straight segment, that is tapered, (iv) A third straight segment, that is not tapered, (v) A first relatively long curved segment, (vi) A second curved segment that assumes a semicircle, (vii) A flipped-tip segment that comprises a 0.5 centimeter straight segment followed by a one centimeter long arc that assumes a 45 degree curve, wherein the flipped-tip segment comprises an aperture that is open to the environment of use, wherein the first curved segment together with the second curved segment assume a J-shaped arc, wherein the second curved segment together with the third curved segment assume an S-shaped arc, wherein the hub defines an axis and wherein each segment defines an axis, and wherein the combined length of the hub plus the first straight segment has a value that is selected from a length that is between 100 cm and 115 cm, wherein the second straight segment, that is tapered, is two centimeters long as measurable along axis of lumen of straight tapered segment, wherein the second straight segment, that is tapered, has a taper that gets narrower when moving from the proximal to distal direction, wherein taper begins at 6 French (outer diameter) and ends at 4 French (outer diameter), wherein the third straight segment, that is not tapered, has a width of 4 French and a length of 2 centimeters, wherein the first curved segment has a constant width of 4 French, and has an arc of 45 degrees, and is about six centimeters long as measurable along central axis of first curved region, wherein the second curved segment assumes a 180 degree semicircular arc, with constant width of 4 French, and wherein the third curved segment comprises the distal terminus of the catheter, and wherein the semicircular arc has an outer circumference diameter of 1.5 cm, and a radius of 0.75 cm, and wherein catheter has a total length measurable along central axis, wherein the total length is either 115 cm or 125 cm, and wherein the distal tip possesses a 4 French outer diameter and 0.038 inch inner diameter, and wherein the distal tip terminates in an aperture that is open to the environment of use.

The distal region of the catheter, which may be called a “shepherd's hook” or a “candy cane hook,” possesses a 45-degree bend (the flipped-tip). This 45 degree bend provides the advantage in some circumstances of providing extra engagement by positioning the distal region of the catheter on the wall of the artery that is most likely to have an origin of the target artery.

What is also embraced it the above catheter that comprises a hub.

Also, what is embraced is the above catheter that does not comprise any hub.

What is contemplated is the above catheter, in combination with a guidewire with a stiff “arch” segment that is about 150 centimeters long, followed in the proximal to distal direction with a tapered region that is about ten centimeters long, followed in the proximal to distal direction, with a tapered segment that is about twenty centimeters long where the taper is from about 0.035 inches down to about 0.012 inches, where the guidewire terminates with a microwire that is about 20 centimeters long, and where this microwire terminates with a tip with diameter of from 0.012 inches to 0.018 inches.

In exclusionary embodiments, the present disclosure can exclude any guidewire that does not include a microwire, and can exclude any guidewire that at its distal-most end has a diameter greater than 0.018 inches in diameter, or has a diameter greater than 0.020 inches in diameter, or has a diameter greater than 0.022 inches in diameter, or has a diameter greater than 0.024 inches in diameter, and so on.

Moreover, what is also contemplated is a kit that comprises the above catheter, wherein the kit further comprises one or more of a balloon tip sheath, a board base, a wrist pad, a grip bar, and a table top.

Alternative design embodiments. Furthermore, the present disclosure provides an alternate design catheter, as defined herein, where in the alternate design catheter comprises a tapered straight region that is about six centimeters long, and that does not include any tapered straight region that is between about two centimeters long and about three centimeters long, wherein the alternate design catheter is: (i) The alternative design of the basic catheter, (ii) The alternative design of the catheter with an S-shaped curve, or (iiii) The alternative design of the catheter with the flipped-tip.

Manufacturing embodiments. What is provided is a method for manufacturing a catheter, wherein the method uses a mandrel, wherein the mandrel comprises polytetrafluoroethylene (PTFE) liner coating, and wherein the mandrel is a scaffold for manufacturing said catheter, the method comprises the steps of: (a) The step of cutting the mandrel to the desired size. (b) The step of placing the mandrel in a machine that comprises spools of stainless steel and nitinol, (c) The step where the machine applies the stainless steel and nitinol coil winds on top of the PTFE liner on the mandrel, in order to form a pattern that is a single wire of stainless steel followed by three wires of nitinol with varying thicknesses, wherein the catheter excludes any coil winding at the distal 1.0 to 1.5 centimeters, (d) The step of repeating the nitinol-stainless steel pattern on the proximal shaft (shaft closest to hub), (e) The step of applying a coil consisting of only nitinol and without any steel, on the distal shaft, (f) The step wherein optionally, nitinol and stainless steel are joined together by laser welding, (g) The step wherein polymers are applied after the metal is applied, wherein polymer sections of varying stiffnesses according to the desired flexibility of the catheter section are placed over the metal, (h) The step wherein said polymers are then bonded to the catheter section underneath it with heat treating, optionally and preferably where it is suspended top to bottom to allow the heat set to bind the coil to the polymer, (i) The step where a hydrophilic coating is applied to the distal section, via dipping with a mandrel inside, usually on the distal aspect, but typically not at the segment that interacts with the arch, approximately 30 mm section, halfway along the catheter, wherein for a preferred design dip the shaft to coat it with the antivasospasm agent, (j) The step wherein optionally, a hub is affixed to proximal end of the catheter, (k) The step wherein, optionally, anti-vasospasm drug in an excipient is applied to the catheter as a coating, wherein excipient can comprise a hydrogel or a time-release formulation or a hydrophilic polymer, and wherein the sum of all segments of catheter comprises catheter shaft, and wherein the coating is applied to entire catheter shaft, or to entire catheter shaft but not to semicircular segment, or to entire catheter shaft but not to flipped-tip and not to semicircular segment, (l) Wherein said method produces a final catheter has three layers from hub to tip, wherein from inside to outside, there is a polytetrafluoroethylene (PTFE) liner section, a metal coil wind section, and a polymer section Typically a hub can be affixed, and the hub is optional.

Coating embodiments. What is provided is the above catheter that comprises a coating, wherein the coating comprises an anti-vasospasm drug, and wherein the anti-vasospasm drug is capable of release in an amount sufficient to reduce the frequency or intensity of spasms of blood vessels when said catheter is inserted into a patient's vasculator and then passed through the patient's vasculature towards the aortic arch, or when at least part of the catheter is inserted into and the patient's aortic arch.

Another manufacturing embodiment. What is provided is the above catheter that is manufactured by the above-disclosed method.

Anti-vasospasm embodiments. What is provided is the above catheter, wherein the catheter comprises a supply of an anti-vasospasm drug, and wherein the catheter is capable of releasing the anti-vasospasm drug from the sheath's sidearm through small channels or through rivulets or through laser-cut holes from the catheter that run along the shaft of the catheter, wherein the channels, rivulets, or holes do not allow blood to enter the sheath.

Sheath embodiments, and catheter/sheath combination embodiments. What is provided is a sheath that comprises a shaft, wherein the sheath is capable of being used to introduce the above-disclosed catheter into the vasculature of a patient, wherein the sheath comprises a supply of an anti-vasospasm drug, and wherein the sheath comprises a sidearm, wherein the sheath is capable of releasing the anti-vasospasm drug from the sheath's sidearm through small channels or through rivulets or through laser-cut holes from the sheath sidearm that run along the shaft of the sheath, wherein the channels, rivulets, or holes do not allow blood to enter the sheath.

METHODS FOR USING IN A PATIENT. The present disclosure provides a method for using the above catheter in a patient, for the treatment or diagnosis of a cardiovascular condition in the patient, the method comprising one or more or all of the steps of: (i) The step of inserting a guidewire into the catheter to form an assembled catheter plus guidewire, followed by inserting the assembled catheter plus guidewire into a sheath, to form an assembled guidewire plus catheter plus sheath, (ii) The step of inserting the catheter into a sheath, to form an assembled catheter plus sheath, followed by inserting a guidewire into the catheter, to form an assembled guidewire plus catheter plus sheath, (iii) The step of inserting the assembled guidewire plus catheter plus sheath into the patient's vasculature wherein the inserting is at the radial vasculature or at the femoral vasculature, (iv) The step of pushing at least the catheter of the assembled guidewire plus catheter plus sheath into the patient's aortic arch.

MODULAR POSITIONING SYSTEM (MPS) EMBODIMENTS. The present disclosure provides a system for transradial access of the vasculature, which comprises, in combination: a plurality of specialty shaped and formed catheters; a grouping of compliant balloons, sheaths and wire tools; and wherein said catheter lengths are ranging between at least about 111 and 127 centimeters; having approximately 3.5 to 5.5 French diameters throughout.

In another aspect, what is provided is the above system, and the entire disclosure, further comprising: at least a curved inner catheter optimized for selection of the origins of the arteries in the body.

In yet another aspect, what is provided is the above system, and the entire disclosure, further comprising: the plurality of specialty shaped catheters being coaxially introduced inside of another catheter which is lubricious and having compliance in certain segments for advancement.

Moreover, what is embraced is the above system, wherein each of the plurality of catheters are introduced simultaneously or in alternating fashion to advance into a select artery after initial engagement.

Further contemplated is the above system, and the entire disclosure for providing a means for accessing the endoluminal cerebral vasculature from the radial artery.

In another system embodiment, what is provided is a novel enhanced system for transradial cerebral access, as shown and described herein, and different from known systems, comprising in combination, at least a kit further comprising at least three sets of specialty catheters; a wire, sheath and introducer in predetermined size ranges, and methods for optimizing combination of catheters and said other tools.

Moreover, what is provided is a Modular Positioning System (MPS), as shown and described, further comprising, in combination a board base, a grip bar; and a wrist pad.

In another aspect, what is provided is the above modular positioning system of the figures and entire disclosure as shown and described is a table-top version. Also provided is an MPS as disclosed further comprising absorbent yet hydrophobic/repellent disposable material.

The disclosure further embraces the MPS as claimed, shown and described, further comprising: wrist pad locators, with attachment sections; ledges; and a plurality of apertures. In another aspect, what is provided is the above MPS, shown and described that is capable of facilitating radial access. Also, what is provided is the above MPS, shown and described, that is capable of general vascular access. Moreover, what is provided is the above MPS, shown and described that is capable of transradial cerebral access.

KIT EMBODIMENTS. The present disclosure provides a kit, comprising transradial access tools including a set of curve specific catheters, introducers and specialized wires. Also provided is the above kit, further comprising an MPS. Moreover what is provided a kit that is customized for transradial cerebral access.

TRANSRADIAL ACCESS SYSTEM EMBODIMENTS. What is provided is a transradial access system, comprising, in combination: a non-transfemoral approach to neuro-endovascular procedures comprised of catheter assemblies ranging from at least about 111 to 127 cm's, with Fr sizes between at least about 3.2 to 5.9; a plurality of specialized curves emplaced within said catheter assemblies; specialized wires, sheaths and introducers. Also provided is the above system that is capable of cerebral access. In another aspect, what is provided is the above system that is capable of use in geriatric patients, or that is capable of patients with unwound (aged) aortic arch, being further specialized, customized and adapted to challenging blood vessel morphologies.

Disclosures from Text Printed on Original Drawings.

Original drawing of the FIG. 5 catheter. Length of the hub plus the first straight catheter segment can be, 103.8 cm or 113.8 cm, as printed on original drawing. These lengths are disclosed in original drawing of the FIG. 5 catheter, where these lengths were called, “proximal catheter.” Outer circumference of semicircular distal-end curve (the end farthest from hub) is 1.5 centimeters in diameter.

Original drawing states that total catheter length is 215 centimeters. But original drawing also states total catheter length is 115 centimeters or 125 centimeters.

Original drawing of FIG. 5 states that distal catheter length is 11.2 cm and that proximal catheter length is either 103.8 cm or 113.8 cm. Original drawing states that: 6 F long sheath specs: 110 cm and/or 100 cm. Regarding the straight tapered segment, this can take a first design or it can take an “alternative design.” Original drawing of FIG. 5 states that first design is three centimeter taper that is followed by three centimeter straight segment, and that alternative design is six centimeter taper, where this tapered segment is not followed (in traveling away from hub, and along central axis of catheter) by any straight segment. In other words, the basic design catheter can exclude any type of catheter that has a straight tapered segment, where this straight tapered segment is followed (in traveling along axis away from hub) by a straight segment.

Original drawings of the FIG. 6 catheter (catheter with S-shaped region). Length of the hub plus the first straight catheter segment can be, 103.8 cm or 113.8 cm (labeled in original drawing as proximal catheter length). Original drawing of FIG. 6 (catheter with S-shaped region) states that alternate design of catheter with S-shaped region has a six centimeter straight taper (and does not have the three centimeter taper of 6 French down to 4 French followed by proximal straight segment of three centimeters that is shown in the non-alternative design of the catheter with S-shaped region.

UNIQUE DIMENSIONS FOR THE “ALTERNATIVE DESIGN” EMBODIMENTS DISCLOSED BY EACH OF FIG. 5 (basic design), FIG. 6 (S-shaped curve), and FIG. 7 (flipped-tip). Each of FIG. 5, FIG. 6, and FIG. 7 of the original drawings discloses a non-alternative design and an “alternative design.”

In FIG. 5, the non-alternative design has a tapered segment followed by a straight segment, but the Alternative Design has a single 6 cm tapered segment that replaces the 3 cm tapered straight segment and the 3 cm straight segment of the non-alternative design.

In FIG. 6, the non-alternative design has a 3 centimeter tapered segment that tapers from 6 French to 4 French, whereas in contrast, the Alternative Design has a six centimeter tapered segment. The only difference between the non-alternative design and the Alternative Design is the length of the tapered segment

In FIG. 7, the non-alternative design has a two centimeter long tapered segment that is followed by a two centimeter long straight segment. In the Alternative Design of the FIG. 7 catheter, the two cm tapered segment and the two centimeter straight segment are deleted, and then replaced with a six centimeter tapered segment.

Original drawings of the FIG. 7 catheter (catheter with flipped-tip). The three curved segments, going from proximal end (end that is closest to hub) to distal end (end farthest from the hub), are a six centimeter long curved segment with a 45 degree arc, followed by 0.75 centimeter radius semicircle, which is followed by the flipped-tip. Flipped-tip in the non-alternative design and flipped-tip in the Alternative design takes the form of a 0.5 cm straight segment that is followed by a curved segment that has 45 degree arc.

The curved segment of the flipped-tip and the curved segment of the six centimeter-long curved segment have a CENTRAL POINT that resides on the same side of the central axis of the catheter with the flipped-tip (the term, “same side” means that the CENTRAL POINTS of the curved segment of the flipped-tip and the curved segment with the six centimeter-long cured segment are near each other. In contrast, if the term had been “different side,” the CENTRAL POINTS of each of these curved segments would be relatively far away from each other).

Dimensions that are shared completely or shared partly by original drawings of FIG. 5, FIG. 6, and FIG. 7 catheter. Total catheter length, 115 cm or 125 cm. Each of these three original drawings have this same, identical writing: “6 F long sheath specs: 110 cm and/or 100 cm. 6 F (0.087 inch min) ID/7 F (0.099 inch max) OD.” ID is internal diameter, and OD is outer diameter. Each of these three original drawings also have this same, identical writing: “Alternative design for diagnostic angiography: Introducing catheter specs. 4 F entire length. Long sheath specs 4 F ID/5 F OD.”

Also, the first two of these three original drawings have the same writing about the proximal catheter (proximal catheter consisting of hub plus first straight segment): “Proximal catheter length 103.8 centimeters or 113.8 centimeters.” And the third of these three drawings (FIG. 7) says, “Proximal catheter length 101.3 centimeters or 111.3 centimeters.” The first two of these three drawings also have their own, unique, distal catheter length, where this proximal distal catheter length is not shared by the third drawing (FIG. 7). The first two of these drawings say, “Distal catheter length 11.2 centimeters” but the third drawing (FIG. 7) says, “Distal catheter length 13.7 centimeters.”

GLOSSARY. In directionality embodiments of the system, medical device, catheter, sheath, combination of catheter and sheath, guidewire, combination of guidewire and catheter, and methods of the present disclosure, the term “proximal” refers to regions of medical device that is closest to end of catheter that has a hub, while the term “distal” refers to region that is farthest away from hub.

As a non-limiting glossary term, where terms such as, “first segment” and “second segment” are used, the word “segment” can be used to distinguish two different segments from each other, where the “first segment” and “second segment” are distinguishable from each other because they are separated by a discernable transition point that is gradual, or by a discernable transitional point that is sudden, where the transition takes one of the following forms. The transition can take the form of a change in French value of the first segment and of the second segment. The transition can take the form of a change in curvature between the first segment and the second segment, for example, where the first segment is straight (not curved at all), and where the second segment assumes a curved arc.

Also, terms such as, “first segment” and “second segment” can also be used to refer to catheter structures, or to sheath structures, where the first segment is coated with a first type of chemical composition and the second segment is either not coated or is coated with a second type of chemical composition.

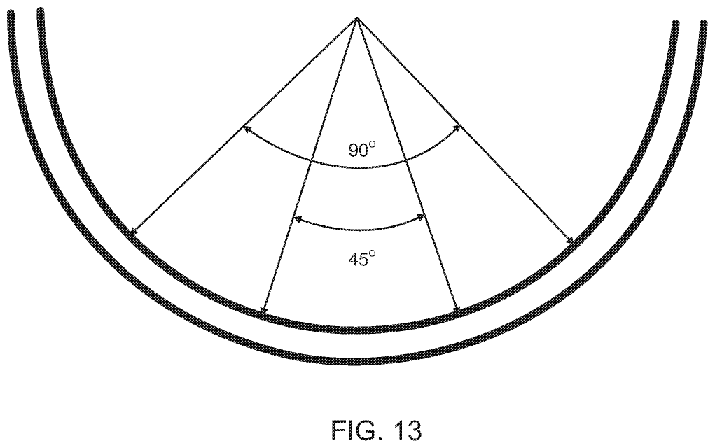

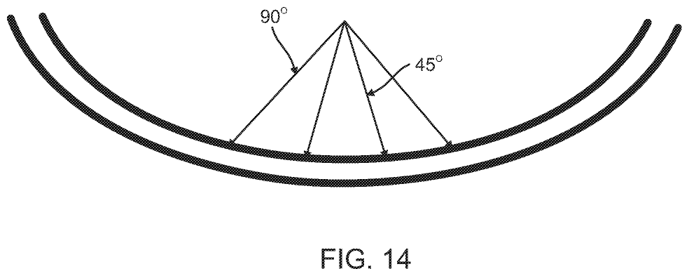

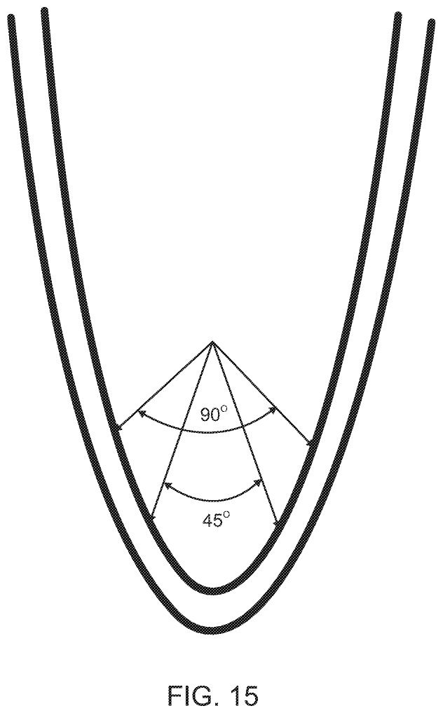

The term, “curved segment” can refer to a segment of a catheter or other medical device, that has a curve definable by region cut out from the arc of a circle (see, e.g., the circle of FIG. 13). Also, “curved segment” can refer to a segment of a catheter or other medical device that assumes a curve definable by a region cut out from the arc of the squashed and substantially flattened region of an oval. See, the squashed and substantially flattened region shown of the oval of FIG. 14. Also, “curved segment” can refer to a segment of a catheter or other medical device that assumes a curve definable by a region cut out from the arc. See, the squashed and substantially scrunched-up region of the oval of FIG. 15. Preferably, the term “curved segment” is never used to refer to a segment of a catheter or other medical device, where the

Flipped-tip segment residing at the distal end of catheter. Any catheter embodiment of the present disclosure can include, at the distal end, an additional segment (segment that is even more distal) taking the form of a flipped tip. Flipped-tip consists of a short straight segment that is coupled to a short curved segment. Curved segment assumes a curve that defines a 45 degree angle, and where the curve that assumes a 45 degree angle that has a central point that is near to (rather than, relatively far away from) a long curved segment that is immediately proximal to the flipped-tip. In other words, flipped-tip is distal and long curved segment is proximal, where “distal” means relative far from the hub, and “proximal” means relative close to the hub.

In catheter embodiments that do not include any hub, the term “proximal” can refer to the end of the catheter that is farther from patient's heart immediately after the catheter is inserted into patient's vasculature, and the term “distal” can refer to the end of the catheter that is closer to patient's heart immediately after the catheter is inserted into patient's vasculator.

### DETAILED DESCRIPTION

Traditional open vascular approaches, in which a surgeon makes an incision into the skin and exposes the vessel in order to make surgical corrections or alterations, are being replaced by endovascular procedures. These more modern, minimally invasive approaches begin with only a needle puncture in the skin, following which catheters or small, elongated tubes are advanced into the body's vessels and navigated throughout the body's blood vessel system, or vasculature, using imaging in order to deliver the therapy. Endovascular access products such as catheters and wires are essential in order to achieve access to any specific area in the vasculature. Access products require different physical properties depending on multiple factors: location of access, individual dimensions and conformation of the patient's vasculature, as well as the exact route and target of intravascular navigation. A patient's age has a significant impact on a patient's vasculature's conformation.

Endovascular techniques are predominantly driven by specialists based on organ system and have been refined by them as techniques evolve. For instance, initial arterial endovascular approaches were done by puncturing the neck artery. Due to the potential for injury to that vessel, endovascular approaches subsequently developed into puncturing the groin artery (transfemoral approach). For doctors addressing the arteries of the heart, over the past decade the majority have been accessing across the wrist artery (transradial approach), as the approach is more direct, substitutes the higher risk of groin access complications for the lower risk of wrist issues, and leads to increased patient satisfaction and shorter, improved post-procedural recovery.

Neuro-endovascular procedures, those dealing with the brain's vasculature, still predominantly utilize the transfemoral approach. The majority of neuro-endovascular procedures are performed in the younger and middle-aged population, lending to the ease of the transfemoral approach to access all the vessels of the head and neck. Due to the advancing patient age from the increasing number of acute stroke therapies, the vasculature conformation is increasingly becoming more difficult via the transfemoral approach. Concomitantly, the need for larger catheters and the increasing use of stronger blood thinning agents (agents that impair blood clotting) required for some procedures, both increase the life-threatening nature of groin access complications.

Transradial approaches for neuro-endovascular procedures are still in their infancy. As the majority of transradial access products are optimized for navigation to the cardiac vasculature, there is need for optimized products for transradial navigation to the insides of the cerebral vessels, or endoluminal cerebral vasculature.

Described herein there is shown a system and method of use for endovascular access to the cerebral vasculature from a transradial approach. And in one embodiment, a curved, inner catheter that is optimized for selection of the origins of the arteries in the neck and coaxially introduced (placed inside) another catheter which is optimized to be lubricious so as to smoothly interact with the insertion point in the skin, and compliant in certain segments to allow for advancement over the curved platform created by the introducing catheter. In one method, the outer catheter is advanced over the platform. In another method, the introducing catheter and outer catheter are used simultaneously or in an alternating fashion to advance into a selected artery after initial engagement. In one embodiment, this outer catheter is a sheath. In another embodiment, this outer catheter is a guiding catheter. In one embodiment, the outer catheter is optimized to be have a small outer diameter in order to be minimally occlusive in the smaller wrist artery, yet having a large inner diameter, being able to accommodate the larger catheters needed for neuro-endovascular procedures. Thus, the dimensions of the outer catheter are optimized to provide this. In one embodiment, the outer catheter is optimized with varying segments of stiffness and compliance along its length to accommodate for the different positions, angles of strain and forced curvature when achieving and maintaining conformation across the vasculature course.

Using the framework of an apparatus and system comprised of one or several optimized catheters, and a method described herein, the aim is to provide a means to access the endoluminal cerebral vasculature from the radial artery.

Systems, devices, and methods of the present disclosure can navigate most or all variants and geometries of the aortic arch, in particular, when approaching in a retrograde fashion from the right radial artery.

### Advantages of Systems, Devices, Catheters, and Methods of the Present Disclosure

Without implying any limitation, the present disclosure provides a number of advantages.

Advantages of each of the inventive curves and shapes of the catheter are that they allow for optimal positioning to make the turns from the right subclavian artery into the right common carotid artery or the bovine left common carotid artery; alternatively from the innominate artery into the arch then back up into the left common carotid artery or the left subclavian artery; furthermore, to provide a stable tip curve to first allow a wire to be advanced through the catheter up as high as needed, then taking advantage of the design optimization in its design-in-totality with the wire stiffness and diameter once the wire is fully advanced, can then be advanced over the wire until the catheter's tip is in a desired location in the neck artery.

Another advantage is reduced adverse events, including reduced neurological adverse events. These include reduced neurological adverse events due to scraping of the top of aortic arch where calcium and cholesterol tend to deposit, where the scraping occurs when advancing a catheter trans-femorally up into vessels that pass through vessels residing in the neck and in the brain.

Another advantages of the present catheter over existing catheters, is that present catheter has (1) Smaller outer diameter relative to larger inner diameter; (2) Lubricity of the present catheter that allows for smoother interactions, especially when catheter passes through vessels that are smaller and more spastic, relative to the large femoral artery; (3) Release of anti-vasospasm drugs by the present catheter, which also contributes to smoother interactions with blood vessels, especially when catheter passes through vessels that are smaller and more spastic, as compared to the large femoral artery.

ADVANTAGE OF THE FLIPPED-TIP. The distal region of the catheter, which may be called a “shepherd's hook” or a “candy cane hook,” possesses a 45-degree bend (the flipped-tip). This 45 degree bend provides the advantage in some circumstances of providing extra engagement by positioning the distal region of the catheter on the wall of the artery that is most likely to have an origin of the target artery. ADVANTAGE OF THE S-SHAPED CURVE. The S-curved region allows for an easier engagement of the arteries from a radial approach, specifically the right carotid and a bovine left carotid.

Sheath of the present disclosure, advantages, methods of manufacturing. Sheath has different transition zones based on the length coming from a radial approach. The zones of stiffness provide support across the aortic arch or curve into the carotids, while they also are the least hydrophilic, as they do not need to be slippery in these areas. Slipperiness allows more freedom of movement and thus lends to instability of the delivery platform.

Methods for Manufacturing Catheter of the Present Disclosure.

The present invention comprise a method for manufacturing catheter. The main components of the catheter include, a tube made of a polymer, a coil winding, a mix of stainless steel, and a nitinol hydrophilic coating.

For manufacturing, it all starts with the mandrel. The mandrel is like a scaffold. Mandel has a PTFE liner coating on it. The mandrel is cut to size and placed in a machine with spools of stainless steel and nitinol. The machine applies the stainless steel and nitinol coil winds on top of the PTFE liner on the mandrel. The pattern is a single wire of stainless steel followed by three wires of nitinol with varying thicknesses (this is one embodiment only). The catheter is typically without coil winding at the distal 1-1.5 cm. This nitinol-stainless steel pattern repeats on the proximal shaft. At the distal end of the catheter, the construction is completely nitinol. There may also be laser welding joining nitinol and stainless steel. Once the metal is applied correctly, the polymers are applied. Polymer sections of varying stiffness according to the desired flexibility of the catheter section are placed over the metal. These polymers are then bonded to the catheter section underneath it with heat treating (usually it is suspended top to bottom to allow the heat set to bind the coil to the polymer). A hydrophilic coating is applied to the distal section, via dipping with a mandrel inside, usually on the distal aspect, but typically not at the segment that interacts with the arch, approximately 30 mm section, halfway along the catheter; for our design, we may need to dip the shaft to coat it with the antivasospasm agent (see below). The final catheter has three layers from hub to tip. From inside to outside, there is a PTFE liner section, a metal coil wind section, and a polymer section. Typically a hub can be affixed, and the hub is optional.

In exclusionary embodiments, system, medical device, catheter, and methods of the present disclosure can exclude any medical device that comprises, stainless steel, nitinol, tantalum, ceramic, nickel, titanium, aluminum, polymeric materials, stainless steel, titanium, niobium, or gold.

Coating methods; chemical reagents for coating. Coating can be via spray-coating, dip-coating, brushing, or vacuum-deposition.

Unique Geometry of the Catheter.

A unique aspect of the catheter is that there is a variable geometry to the catheter to allow for the two different optimal conformations. The sheath and catheter are optimized and minimized difference between the inner diameter and the outer diameter. Manufacture as mentioned above may allow this to occur without sacrificing catheter strength and trackability.

Methods for Inserting Catheter into Vascular System of a Patient.

Insertion via the arteries in the wrist, radial artery as an example, to be advanced up the arm in a retrograde fashion (against the direction of blood flow), down into the upper thorax to then select the arteries of the head and neck in an anterograde fashion (the direction of blood flow). Use of the long sheath or balloon guiding catheter are similar, with the intent to be guided into and positioned running the length from the wrist to high into the neck to allow for a stable platform for insertion of devices for intervention.

Using a Guidewire.

Having a distal wire tip that is optimized/minimized in its outer diameter for insertion farther into the artery as it enters into the head/skull, while having a much larger transition to a larger diameter shaft, to present a stiff approach as it makes a turn in the arch or above it (as in a bovine left carotid artery), over which a catheter and/or sheath can be advanced over it without concern of “herniating” (or translating the pushing forces) the catheter out further into the arch, which is a hazard.

Coating of Anti-Vasospasm Drug on the Catheter's Shaft.

The anti-vasospasm aspect of the catheter is in one embodiment a coating that prevents vasospasm, and lies over or in conjunction with the hydrophilic coating along any if not most of the shaft, not necessarily the tip. The drug can also be in a hydrogel. The drug can have the molecule coating the catheter. The drug can also be injected from the sheath sidearm and pass through a small channel(s) or rivulet(s) or laser cut holes (that allow injected drug from the sheath sidearm to to pass through the laser cut holes but does not allow blood to enter) running along the shaft of the catheter or sheath. An alternate embodiment of the anti-vasospasm drug takes the form of a time-released formulation.

Coating can be made from, for example, polyesters, polyimides, nylons (polyamides), polytetrafluoroethylene (PTFE), ethylene chlorotrifluoroethylene (ECTFE), perfluoroalkoxy (PFA), polyethylenes, polypropylenes, polyether block amide (PEBA) such as a Pebax®, polyurethanes, and so on. In exclusionary embodiments, system, medical device, catheter, of the present disclosure can exclude any system, medical device, or catheter that comprises one or more of the above chemicals.

Coating and Impregating Medical Device.

The present disclosure provides a formulation for applying to a surface of a medical device, for example, by soaking, where the formulation comprises a dissolved plastic polymer. The dissolved plastic polymer can be more or more of or any combination of, polyurethane, polyethylene, polyethlyene teraphthalate, ethylene vinyl acetate, silicone, tetrafluoroethylene, polypropylene, polyethylene oxide, polyacrylate, and so on. What is encompassed are coatings, coating solutions, and medical devices that are coated with coating solutions, using Carbothane® family of polycarbonate-based aliphatic and aromatic polyurethanes, Estane®, which is a thermoplastic polyurethane, Pellethane®, which is a family of medical-grade polyurethane elastomers and exceptionally smooth surfaces, Tecoflex®, which is a family of aliphatic polyether polyurethanes, where low durometer versions are particularly suitable for long-term implant applications, Tecothane®, an aromatic polyurethane, Texin®, an aromatic polyether-based polyurthane which allows for very thin gauges (Microspec Corp., Peterborough, N.H.; Lubrizol, Inc., Wickliffe, Ohio; Entec Polymers, Orlando, Fla.). See, U.S. Pat. No. 6,565,591 of Brady, U.S. Pat. No. 7,029,467 of Currier, and U.S. Pat. No. 7,892,469 of Lim, which are incorporated by reference in their entirety. In embodiments, the present disclosure provides the recited polymers for use in coating solutions, or for use in manufacturing the medical device that is to be coated. A reagent, such as an anti-vasospasm agent, can be bulk distributed in the medical device, for example, by adding to a melted polymer or by soaking until even distribution has occurred.

Alternatively, medical device can be impregnated or coated with the agent. In embodiments, the disclosure encompasses methods for bulk distribution, gradient distribution, and limited surface distribution. Methods for manufacturing medical devices where an agent is bulk distributed, gradient distributed, or limited surface distributed, are available (see, e.g., U.S. Pat. No. 4,925,668 issued to Khan, et al, U.S. Pat. No. 5,165,952 issued to Solomon and Byron, and U.S. Pat. No. 5,707,366 issued to Solomon and Byron, all of which are incorporated herein by reference).

Coating and Impregnation.

Generally, coating resides on, or adheres to, the exterior surface of medical device. Coating thickness can be, about 10 nanometers (nm), about 50 nm, about 100 nm, about 500 nm, about 1.0 micrometers (um), about 10 um, about 50 um, about 100 um, about 500 um, about 1 millimeters (mm), about 5 mm, and so on. Material used for coating can extend into the medical device, and this aspect of the coating can be referred to as an impregnation. Impregnation can extend throughout entire medical device, and where extension throughout device is substantially uniform, the impregnation is a bulk distribution. Impregnation can extend, without limitation, about 10 nanometers (nm), about 50 nm, about 100 nm, about 500 nm, about 1.0 micrometers (um), about 10 um, about 50 um, about 100 um, about 500 um, about 1 millimeters (mm), about 5 mm, and so on, from the surface into medical device. Alternatively, device can be manufactured so that an agent does not reside on the surface, but resides only in interior of medical device. Use of the term “coating” or “impregnation” can depend on whether the coating or the impregnation is functionally more important.

Measuring flexibility of entire catheter or of a segment of catheter. Flexural Modulus determines how much a sample will bend when a given load is applied, as compared to Tensile Modulus which determined how much a sample will stretch when a given load is applied and Compressive Modulus which determines how much a sample will compress when a given load is applied. Procedures for testing flexural modulus include, ASTM D790 and ISO178 (see. Beetle Plastics (Apr. 11, 2013) Testing and measuring flexural modulus. Ardmore, Okla.). Flexural Modulus by ASTM D790 or by ISO78 can be measured using a ZwickRoell testing machine (see, ZwickRoel, Kennesaw, Ga.). In embodiments, entire catheter, or a given segment of catheter of the present disclosure can have a Flexural Modulus of about 2 kpsi, about 4 kpsi, about 6 kpsi, about 8 kpsi, about 10 kpsi, about 12 kpsi, about 14 kpsi, about 16 kpsi, about 18 kpsi, about 20 kpsi, about 22 kpsi, about 24 kpsi, about 26 kpsi, about 28 kpsi, about 30 kpsi, about 32 kpsi, about 34 kpsi, about 36 kpsi, about 38 kpsi, about 40 kpsi, about 60 kpsi, about 80 kpsi, about 100 kpsi, about 120 kpsi, about 140 kpsi, about 160 kpsi, about 180 kpsi, about 200 kpsi, about 220 kpsi, about 220 kpsi, about 240 kpsi, about 260 kpsi, about 280 kpsi, about 300 kpsi, about 320 kpsi, about 340 kpsi, about 360 kpsi, about 380 kpsi, about 400 kpsi, or the Flexural Modulus can take the form of a range that is bracketed by any of the above two values. The word about, can mean plus or minus 5 percent, plus or minus 10 percent, plus or minus 15 percent, plus or minus 20 percent, plus or minus 50 percent, and so on.

Catheter, proximal segment (segment that is closer to hub), distal segment (segment that is farther from hub), straight segment, S-curved segment, semicircular segment, curved segment, sheath, or any segment of sheath, of the present disclosure can be manufactured so that it possesses a can also possess a Flexural Modulus that can match one of the values, or can be within one of the ranges, that are disclosed above. Also, any of these segments can exclude embodiments that possess one of the values that match one of the above values, or that is within one of the ranges that are disclosed above.

Catheters of the present disclosure also encompass catheters as described therein (where the description includes a hub), but where the catheter does not include any hub, for example, where the same description applies but where the word “hub” is deleted, or where the word “hub” is replaced with a phrase, such as, “region of catheter that is farthest away from semicircular segment. Both types of catheters (with or without hub) are encompassed.

In exclusionary embodiments, system, medical device, and catheter of the present disclosure can exclude any system, medical device, or catheter, where a catheter or segment thereof, a tubing or segment thereof, a sheath or a segment thereof, is definable by one of the above Flexural Modulus parameters.

Measuring hardness of entire catheter, or of a specific segment of catheter, or of transition region between two adjacent regions of catheter, or of sheath. Hardness of a plastic can be defined in terms of a “durometer” value. Hardness is defined and tested as a material's resistance to indentation. The hardness can be, for example, about 45, about 50, about 55, about 60, about 65, about 70, about 75, about 80, about 85, about 90, about 95, or about 100. In attributing any of these durometer values to a plastic substance or other substance, one must also state which scale is used. For example, the scale can be ASTM D2240 type A scale, which is used for softer materials, or the ASTM D2240 type D scale, which is used for harder materials (see, Silicon Design Manual, 6.sup.th ed., Albright Technologies, Inc., Leominster, Mass.).

The hardness of the devices of the present disclosure, including hardness of specific features, such as a tip, wall, bump, tapered region, hub, wing, tab, conical region, bead-like region, can be measured by the durometer method and Shore hardness scale. See, e.g., U.S. Pat. No. 5,489,269 issued to Aldrich, U.S. Pat. No. 7,655,021 issued to Brasington and Eleni (2011) Effects of outdoor weathering on facial prosthetic elastomers. Odontology. 99:68-76, which are each individually incorporated herein by reference in their entirety. Shore A hardness refers to hardness determined where a steel rod dents in the material, while Shore D hardness refers to hardness that is determined where a steel rod penetrates into the material. Shore hardness, using either the Shore A or Shore D scale, is used for rubbers/elastomers and is also commonly used for softer plastics such as polyolefins, fluoropolymers, and vinyls. The Shore A scale is used for softer rubbers while the Shore D scale is used for harder rubbers. Hardness by either scale can be measured with instruments from Kraiburg TPE, Buford, Ga., where durometer values can be either DIN53505 standard, or by ISO7619-1 standard.

In embodiments, system, device, medical device, catheter, catheter segment, sheath, or sheath segment, of the present invention can have durometer value of, for example, about 45, about 50, about 55, about 60, about 65, about 70, about 75, about 80, about 85, about 90, about 95, or about 100, or, less than 45, less than 50, less than 55, less than 60, less than 70, less than 75, less than 80, less than 85, less than 90, less than 95, less than 100, or more than 45, more than 50, more than 55, more than 60, more than 65, more than 70, more than 75, more than 80, more than 85, more than 90, more than 95, more than 100, and so on.

In exclusionary embodiments, medical device, catheter, catheter segment, sheath segment, can exclude an medical device, can exclude any catheter, can exclude any sheath, that has a segment where the durometer value is describably by one of the above ranges.

Medical Instruments or Medical Tools that can be Passed Through the Catheter Versus Medical Instruments that are Mounted on an End of the Catheter.

Balloons, fabrics for balloons, layers, adhesives, housings for balloons, devices for inserting and withdrawing balloons, related devices such as stents and catheters, methods of manufacture, and methods for administration, treatment, or diagnosis, and methods for insertion or withdrawal of a medical device from a patient, are available. See, for example, U.S. Pat. No. 7,862,575 of Tal; US 2007/0060882 of Tal, US 2011/0160661 of Elton; US 2010/03180 of Pepper). Each of these patents and published patent applications is hereby incorporated by reference as if set forth herein in its entirety.

In an exclusionary embodiment, system, medical device, catheter, of the present invention can exclude any instruments that are mounted on, or attached to, or coupled to, at the end of the catheter. This system is allowing access to the cerebrocervical vasculature so that tools can be inserted into the catheter or sheath lumen to then entire the brain vasculature to perform the intervention. In exclusionary embodiments, system, medical device, catheter, and methods of the present disclosure can exclude any system, medical device, or method, where a medical instrument or tool is mounted on distal end of the catheter, or where a medical instrument or tool is mounted on proximal end of catheter, or where a medical instrument or tool resides within the lumen of a catheter.

Neurological Adverse Events.

Neurological adverse events, for example, during aortic valve implantation, can include stroke, embolic stroke, atheromatous emboli in carotid arteries, calcific emboli in carotid arteries, hypoperfusive ischemia (see, Grewal, Solometo, Bavaria (2012) Royal College of Surgeons inreland Student Medical Journal 5:12-17), motor deficits due to ischemia in the brain (see, Bergeron, Coulon, Mariotti (2006) Eur. J. Vascular Endovascular Surgery. 32:3845), and spinal cord ischemia (see, Setacci, Chisci (2009) HSR Proc. Intensive Care Cardiovasc. Anesth. 1:37-44).

Medical device, catheter, sheath, assembled catheter and sheath, and methods of the present disclosure reduce neurological adverse events to less than 90%, less than 80%, less than 70%, less than 60%, less than 50%, less than 40%, or less than 30% of the frequency that is associated with medical devices, catheters, and methods, with medical devices, catheters, and methods that were available prior to filing date of the present disclosure, or that were available prior to any date without regard to the present disclosure.

This adverse event frequency can be with respect to a time frame associated with a given medical procedure in a given patient, for example, the time frame beginning with the time and date that medical device is inserted into patient's aortic arch and ending at one hour after insertion procedure was initiated, or ending 5 hours, 10 hours, two days, 5 days, one month, 2 months, 4 months, or ending an indefinite period of time, or ending at an unspecified length of time, or beginning the moment that medical device is inserted into patient's aortic arch or after the moment when the catheter of the present disclosure is initially inserted into patient's vasculature, and ending at one hour after the insertion procedure was initiated, or ending 5 hours, 10 hours, two days, etc., after insertion was initiated.

Embodiments of the Present Disclosure that Comprise Seldinger Technique.

Methods for inserting a catheter or sheath into a blood vessel include the use of the Seldinger technique. The Seldinger technique includes the initial step of inserting a needle into a patient's blood vessel. A guide wire is inserted through the needle and into the vessel. The needle is removed, and a dilator and sheath combination are then inserted over the guide wire. The dilator and sheath combination is then inserted a short distance through the tissue into the vessel. The combination of the needle, dilator, and sheath, can be advanced over the guide wire into the blood vessel. After this combination has been advanced, the dilator is removed. The catheter is then inserted through the sheath into the vessel to a desired location. The Seldinger technique, and variations thereof, and devices used to perform this technique, are described in Seldinger (1953) Acta Radiologica 39:368-376; U.S. Pat. No. 7,722,567 issued to Tal, U.S. Pat. No. 7,972,307 issued to Kraus, et al, and U.S. Pat. No. 7,938,806 issued to Fisher, et al, which are incorporated by reference. U.S. Pat. No. 6,004,301 issued to Carter, incorporated by reference in its entirety, provides several elementary diagrams that disclose the insertion of a needle through the patient's flesh, with insertion into a blood vessel.

Exclusionary embodiments relating to Seldinger technique and electronic components. In embodiments, system, devices, medical devices, catheters, and methods can exclude any system, medical device, or method that uses Seldinger technique, or that comprises a sheath, or that comprises a guide wire.

Alternatively, embodiments, system, devices, medical devices, catheters, and methods can exclude any system, medical device, or method that uses Seldinger technique, or that comprises a sheath, or that comprises a guide wire, for purposes of the invention as defined by the present claim set, but where one or more of Seldinger technique, sheath, and guidewire can optionally be utilized by an operator, where the operator is using system, medical devices, catheters, and methods of the present disclosure.

In exclusionary embodiments, system, devices, catheters, and methods of the present disclosure can exclude electronic components, such as a battery, capacitor, light emitting diode (LED), heating element, motor, radio transmitter, electric wire, coaxial cable, electromagnet, and so on, for the purposes of the invention as defined by the present claim set, but one or more electronic components can optionally be utilized by an operator, where the operator is using system, medical devices, catheters, and methods of the present disclosure.

Preferred embodiments of catheter of the present disclosure. Preferred embodiments of medical device, catheter, or sheath of the present invention, have lubricity, and have anti-spastic coating, each of which contributes to smoother interactions with blood vessels. Lubricity and anti-spastic coating (e.g., coated with agent that prevents vasoconstriction) promote smoother interactions with blood vessels, in particular with blood vessels that are relatively small and more spastic, as compared with the large femoral artery. In addition, preferred embodiments of the catheter have a relatively small outer diameter, and a relatively larger inner diameter, as compared, for example, with commercially available catheters. These relative diameters are detailed below.

Copolymer Embodiments; Porosity Embodiments; Hydrogel Embodiments. Copolymers are encompassed by the disclosure, for example, copolymers of the block type and copolymers of the rake type (see, e.g., U.S. Pat. No. 8,008,407 of Oberhellman et al. and U.S. Pat. No. 8,084,535 of Maton et al, which are incorporated herein by reference in their entirety). Regarding porosity, if the porosity of a polymer coating is not sufficient to allow diffusion of an agent, such as a drug, into the extracellular fluids, a porosigen, such as lactose, can be added to the polymer used for the coating. Hydrogels, and methods for controlling water content of hydrogels, and mechanical strengths of various types of hydrogels are described (see, e.g., U.S. Pat. No. 4,734,097 of Tanabe et al, which is hereby incorporated by reference in its entirety). Because of their weak, rubbery mechanical properties, polysiloxane is sometimes prepared as chemically crosslinked, or synthesized as a block polymer that alternates with a harder type of polymer (see, page 36 of F. Wang (1998) Polydimethylsiloxane Modification of Segmented Thermoplastic Polyurethanes and Polyureas, Thesis, Virginia Polytechnic Institute and State Univ., Blacksburg, Va.)

Lubricity

Silicone can reduce the coefficient of friction. Methods for applying silicone and for measuring coefficient of friction are available (see, e.g., U.S. Pat. No. 5,013,717 of Solomon). What is provided is medical device that retain their “plastic memory,” such as medical device comprising thermoplastic polyurethane, as compared to vinyl resin (see, e.g., U.S. Pat. No. 4,579,879 of Flynn). What is provided is medical device that, in its entirety, or in segments, comprises siloxane. Medical device comprising siloxane has increased flexibility, when compared, for example, to a medical device that is substantially made of polyurethane (see, e.g., U.S. Pat. No. 8,092,522 of Paul et al). “Lubricity” can be quantitated in terms of the unit, coefficient of friction. “Lubricity” measures the frictional properties or tackiness of a material. A low coefficient of friction may be desired for medical devices to minimize trauma to the patient's body (see, page 226 of Vascular Medicine and Endovascular Interventions (ed. by T. W. Rooke) Blackwell Futura (2007)).

Lubricity can be provided by the polymer that is used to manufacture medical device, catheter, or sheath, of the present invention. Silicone can reduce the coefficient of friction. Methods for applying silicone and for measuring coefficient of friction are available (see, e.g., U.S. Pat. No. 5,013,717 of Solomon, which is incorporated by reference in its entirety). What is provided is medical device that retain their “plastic memory,” such as medical device comprising thermoplastic polyurethane, as compared to vinyl resin (see, e.g., U.S. Pat. No. 4,579,879 of Flynn, which is incorporated herein by reference in its entirety). What is provided is medical device that, in its entirety, or in segments, comprises siloxane. Medical device comprising siloxane has increased flexibility, when compared, for example, to a medical device that is substantially made of polyurethane (see, e.g., U.S. Pat. No. 8,092,522 of Paul et al, which is incorporated by reference in its entirety).

Hubs and Couplers of the Present Disclosure.

In embodiments, the present disclosure provides a coupler or lock, which as Luer lock, or unisex Storz type coupler (see, e.g., U.S. Pat. No. 4,602,654 of Stehling et al). Locking tabs are provided (see, e.g., U.S. Pat. No. 5,885,217 issued to Gisselberg et al). Provided is coupler, where one or more radially-oriented protrusions fit into one or more radially-oriented grooves (see, e.g., U.S. Pat. No. 6,336,914 of Gillespie). Locking collar is encompassed (see, e.g., US 2005/0090779 of Osypka). Also provided is coupler, where axially-oriented pin or pins fit into one or more slots (see, e.g., US 2009/0143739 of Nardeo et al). Further provided, is threaded coupler (see, e.g., U.S. Pat. No. 7,422,571 of Schweikert et al). Each of the above patents and published patent applications are hereby incorporated by reference, in their entirety. In embodiments, what is encompassed is a valve, or a medical device that comprises a valve. A coupler can couple a first hub to a second hub, for example, a first hub that is a catheter hub and a second hub that is a needle hub. Or the first hub can be a catheter hub and the second hub can be a sheath hub. Valve of the present disclosure can reside in a housing that is a hub, or the valve can reside in a housing that is not a hub.

Diameters of Catheter and Sheath of the Present Disclosure.

Catheter French size is usually measured according to the outer diameter. Sheath French size is usually a measure of inner diameter, where this inner diameter determines which size of catheter can fit into the sheath. The sheath can have a name, such as, “external introducer sheath.” The French size is three times the diameter in millimeters.

CATHETER EMBODIMENTS. Catheter exemplary internal diameters can be 6 French (F), 8 F, 10 F, 12 F, 14 F, 16 F, 18 F, 20 F, 22 F, 24 F, 26 F, 28 F, 30 F, 32 F, 34 F, 36 F, 38 F, 40 F, and so on. For catheter, exemplary external diameters can be 6 French (F), 8 F, 10 F, 12 F, 14 F, 16 F, 18 F, 20 F, 22 F, 24 F, 26 F, 28 F, 30 F, 32 F, 34 F, 36 F, 38 F, 40 F, and so on.

In exclusionary embodiments, system, medical device, catheter, and methods of the present disclosure can exclude any system, medical device, catheter, and related methods that comprise a catheter that meets one or more of the above French values.

In thickness embodiments for a given catheter, thickness (value of the external diameter minus value of internal diameter) can be, 2 F, 3 F, 4 F, 6 F, 8 F, 10 F, 12 F, 14 F, 16 F, 18 F, and so on, or in the range of 2-3 F, 2-4 F, 3-4 F, 3-6 F, 4-6 F, 4-8 F, 6-8 F, or 6-10 F, and so on.

SHEATH EMBODIMENTS. Sheath exemplary internal diameters can be 6 French (F), 8 F, 10 F, 12 F, 14 F, 16 F, 18 F, 20 F, 22 F, 24 F, 26 F, 28 F, 30 F, 32 F, 34 F, 36 F, 38 F, 40 F, and so on. For catheter, exemplary external diameters can be 6 French (F), 8 F, 10 F, 12 F, 14 F, 16 F, 18 F, 20 F, 22 F, 24 F, 26 F, 28 F, 30 F, 32 F, 34 F, 36 F, 38 F, 40 F, and so on.

In thickness embodiments for a given sheath, thickness (value of the external diameter minus value of internal diameter) can be, 2 F, 3 F, 4 F, 6 F, 8 F, 10 F, 12 F, 14 F, 16 F, 18 F, and so on, or in the range of 2-3 F, 2-4 F, 34 F, 3-6 F, 4-6 F, 4-8 F, 6-8 F, or 6-10 F, and so on.

Exclusionary Embodiments Relating to Catheter Dimensions or to Sheath Dimensions.

In exclusionary embodiments, system, device, catheter, and method of the present disclosure can exclude any catheter that possesses one of the above internal diameters, or any sheath that possesses one of the above internal diameters.

In exclusionary embodiments, system, device, catheter, and method of the present disclosure can exclude any catheter that possesses one of the above external diameters, or any sheath that possesses one of the above external diameters.

In exclusionary embodiments, system, device, catheter, and method of the present disclosure can exclude any catheter that possesses one of the above thicknesses, or any sheath that possesses one of the above thicknesses.

System, device, medical device, catheter, and methods of the present disclosure can exclude any system, device, medical device, and method that uses, encompasses, requires, or expressly makes optional, one or more of a sheath or an introducer or a guidewire

Position along medical device for measuring diameters. For diameter-measuring purposes, medical device can be defined as having an axis, where the axis is centered within the lumen of the medical device. For diameter-measuring purposes, axis can be defined as having a proximal terminus and a distal terminus. Distal terminus is point along axis of catheter that is closer to patient's heart, when medical device is inserted (partially or fully) within artery. Proximal terminus is point along axis that is farthest from patient's heart, when medical device is inserted (partially or fully) within artery. Location of distal terminus and proximal terminus of axis can include all components that contribute to length of catheter or, alternatively, can exclude contribution to axis length, where this contribution is a hub, handle, coupler, bump, bumped tip, arcuate tip, scraping device, guide wire, and so on.

In embodiments, diameter can be measured at a point that is half-way (50%) in between proximal terminus and distal terminus, or at a point that is 30% from proximal terminus and 70% from distal terminus, or at a point that is 40% from proximal terminus and 60% from distal terminus, 60% from proximal terminus and 40% from distal terminus, 70% from proximal terminus and 30% from distal terminus, and the like.

Preferred embodiments for inner diameter and outer diameter of catheter. Advantages of catheter embodiments of the present disclosure include small outer diameter relative to larger inner diameter. For example, this characterization of inner and outer diameter can result in catheter wall thickness of less than 4.0 millimeters (mm), less than 3.0 mm, less than 2.0 mm, less than 1.0 mm, less than 0.8 mm, less than 0.7 mm, less than 0.6 mm, less than 0.5 mm, less than 0.4 mm, and so on. In various embodiments, the above restriction on catheter wall thickness is required for at least 40% of distance between proximal and distal termini, for at least 50%, for at least 60%, for at least 70%, for at least 80%, for at least 90%, or for at least 95%, of the distance between proximal and distal termini.

In exclusionary embodiments, system, device, medical device, catheter, and methods of the present disclosure can exclude any system, device, medical device, catheter, or method that does not meet one or more of the above dimensional parameters.

Transradial Placement of a Catheter

“Transradial approach” of a catheter involves placement of catheter at the radial artery in the wrist or, alternatively, placement in the ulnar artery in the hand or in the forearm or upper arm. The radial artery is a major artery in the forearm. It is a superficial vessel that runs along the lateral, volar aspect of the forearm. It originates in the antecubital fossa as the brachial artery bifurcates into the radial and ulnar arteries. The radial artery perfuses the forearm and hand in conjunction with the ulnar artery (Wallace and Solano (2020) Radial Artery Cannulation. StatPearls Publishing). Doppler ultrasonic imaging can be used to guide cannulation of radial artery (see, Risoe and Willie (1978) Acta Physiologica Scandanavica. 370-378). Medical devices can be inserted into radial artery or into ulnar artery. Ulnar artery may be preferred if radial artery is too small, tortuous, or spastic. Illustrations of anatomy of blood vessels in the arm provide guidance for inserting medical devices (see, Natsis, Totlis, Tsikaras (2006) Folia Morphol. 65:400-405). As an alternative to radial placement, medical devices can be placed in the femoral artery. Catheterization of the right coronary artery or the left coronary artery can be carried out in a procedure where contrast agent is injected into circulatory system.

In exclusionary embodiments, system, medical device, catheter, and methods of the present disclosure can exclude any system, medical device, and method, where catheterization is into femoral artery, into ulnar artery, into radial artery, into any artery that is not radial artery, or into any artery that is not ulnar artery, or into any artery that is not either radial artery or ulnar artery.

ANTICOAGULANTS. Methods and compositions of the present disclosure include anticoagulants, where the anticoagulants prevent the formation of pathological blood clots during insertion of medical device. Anticoagulants include compounds that inhibit one or more steps of the blood clotting cascade or that impair platelet physiology (see, Brody (1999) Nutritional Biochemistry, 2nd ed. Academic Press, London, pages 524-536). Anticoagulants include, aspirin, heparin, clopidogrel, and glycoprotein IIb/IIIa antagonists (such as, tirofaban or eptifibatide). Prior to, during, or after insertion of medical device into an artery, anticoagulant can be injected directly into the artery. Anticoagulant can be injected prior to and during catherization, or during and after catheterization, or all three of prior to, during, and after catheterization. In exclusionary embodiments, the present disclosure can exclude any system, medical device, composition, or method, where anticoagulant administration uses any of the above drugs and methods.

Thrombosis can be prevented by infusion an amount of heparin that is, for example, about 2,000 IU, about 2,500 IU, about 3,000 IU, about 3,500 IU, about 4,000 IU, about 5000 IU, about 5,500 IU, about 6,000 IU, about 6,500 IU, about 7,000 IU, about 7,500 IU, about 8,000 IU, of heparin, for example, of unfractionated heparin. In embodiments, the term “about” can refer to an amount that encompasses the values from the next lowest to the next greatest in above list (this is defined as, 100% of the range). Also, the term “about” can refer to an amount that is centered on the chosen value and that encompasses a range that is 80%, a range that is 60%, a range that is 40%, a range that is 30%, a range that is 20%, a range that is 10%, or a range that is 5% of the range defined as 100% of the range.

In exclusionary embodiments, system, devices, and methods of the present disclosure can exclude any system, device, and method where heparin administration is definable by one or more of the above values or ranges.

Catheter Kickback Occurring with Calcified Aortic Arch.

In embodiments, system, medical device, catheter, and methods of present disclosure can overcome problem of “catheter kickback.” As people age, the aortic arch becomes calcified, elongated, and less compliant. As the arch elongates, it extends beyond the origin of the left subclavian artery, which, along with other supra-aortic great vessels, tethers the aortic ostial segment. This results is greater difficulty in catheterization of these great vessels from a transfemoral approach. Anatomical variations in the aortic arch, such as bovine arch, can complicate access to the great vessels. Arches in aged people also have atherosclerotic plaque posing higher risk of intraprocedural embolic complications. In some patients, tortuosity of the arch and elongation and tortuosity of the great vessels can impair attempts with standard guide wires through recurve catheters result in a catheter “kickback” into the aorta and failure to achieve access (see, Shakir, Siddiqui (2017) Neurosurgical Focus. 42:E14 (5 pages)).

Difficulty in Obtaining Access Caused by Type III Arch. The Steeper the Arch, and the More Inferior the Origin of the Target Artery, there May be Greater Difficulty in Gaining Access to the Target Vessel.

Type I, Type II, and Type III aortic arches have been described according to positions of origin of the supra-aortic arteries: “Aortic arch anatomy can be classified as Type I (all supra-aortic vessels originate at the same level in a straight line), Type II (innominate and left common carotid arteries originate below the left subclavian artery) and Type III (all supra-aortic vessels originate below the straight line, the angle between vessel origin and aortic arch is acute).” (see, Setacci, Chisci (2009) HSR Proc. Intensive Care Cardiovasc. Anesth. 1:37-44).

System, medical devices, catheter, and methods of the present disclosure can overcome difficulties in access and, in particular, difficulties in obtaining access with left common aortic artery. Demertzis (2010) J. Anatomy. 217:588-596, states that Type III arch causes difficulty when trying make a medical device gain access into the “supra-aortic artery.” See also, page 152 of Carotid Artery Stenting: The Basics. Ed. by Jacqueline Saw. Humana Press (2009), which states that, “For carotid intervention, the steeper the arch and in particular the more inferior the origin of the target artery (in Type II or III aortic arches . . . the greater the difficulty in gaining access to the target vessel.” System, medical device, catheter, and methods of the present disclosure overcome these difficulties, by using a catheter with a lubricious coating, where the catheter releases agents that prevent vasospasms, and where the catheter is inserted into femoral artery of the patient.

System, medical devices, catheter, and methods of the present disclosure overcome difficulties in placing a stent, angioplasty balloon, or other medical devices into the carotid artery by allowing ease of access to this artery. These difficulties are disclosed by Popieluszko et al (2017), which concerns placing a stent in an artery. In addition to impairing access by catheters, variations in the aortic arch, such as bovine arch (Type II aortic arch), can, “increase risk for complications during surgical procedures.” For example, bovine arch can make, “carotid stenting more difficult and risky . . . [where] this difficulty may be encountered as a result of a tight turn involving the brachiocephalic trunk (BT) and the common carotid artery (LCC) arteries. (see, Popieluszko, Henry, Sanna (2017) Journal Vascular Surgery. 68:298-306).

Methods for Using Catheter of the Present Invention for Placing Other Medical Devices.

System, medical device, catheter, and methods of the present disclosure find use in placing various medical devices at pre-determined positions in the circulatory system and, in particular, in the aortic arch, or within a chamber of the heart, or within the cerebral vasculature. Locations of placement also include, within the superior cerebellar artery, posterior cerebellar artery, innominate artery, brachiocephalic trunk, aortic arch, subclavian artery, common carotid artery, internal carotid artery, posterior inferior cerebellar artery, anterior inferior cerebellar artery, posterior cerebral artery, middle cerebral artery, Circle of Willis, basilar artery, and within the vertebral artery.

System, medical device, catheter, and methods of the present disclosure find use in placing, e.g., a stent, an angioplasty balloon, a camera, a trocar, and so on. Trocar is a medical device that has these parts: obturator with an optional sharpened tip, a cannula (hollow tube), and a seal. Trocar can be used to perform laparoscopic surgery (this avoids open surgery with a wide incision).

Tortuosity.

This provides a context regarding the anatomy of arteries. This context can enhance understanding of devices and methods of the present disclosure. Most large elastic arteries are stretched longitudinally by traction and follow a straight course. Aged patients and hypertensive patients sometimes develop abnormal curvature (arterial tortuosity) as a result of vessel elongation or as a result of shortening of the distance between arterial tethering points. Compression of the spine in old patients may contribute to increased tortuosity of the abdominal aorta. The curvature of a vessel influences its local flow hemodynamics and may result in unfavourable clinical consequences. For instance, low wall shear stresses are known to occur along the inner curvature of curved arteries may contribute to accumulation of lipids and consequent development of atherosclerotic lesions. The accompanying elevated pressure on the outer walls in a vessel weakened with age may lead to aneurysm, especially in the abdominal aorta (see, Dougherty and Varro (2000) Medical Engineering & Physics. 22:567-574). Tortuosity can be quantitated by one of several different tortuosity indices. For example, one tortuosity index is distance factor (DF). DF equals (LID) minus 1.0. In this formula, L is meandering vessel length and D is straight line distance between end points. For a straight vessel that does not curve and that has no bends, the value of DF equals zero. Dougherty and Varro (2000) have devised an additional tortuosity index that uses data collected (collected from the patient) that represents curvature of an artery in three dimensions. The three dimensional tortuosity index is, TC_{3D}=(TCₓ² plus TC_{y}²)^{1/2}.

Tortuous aortic arch, in patients having a tortuous aortic arch, can cause difficulty in accessing blood vessels in the head and neck (supra-aortic blood vessels), when using the trans-femoral route of accessing the aortic arch. Tortuous aortic arch is described by Wagdi (2013) Cardiol. Res. 4:8-14.

System, medical devices, catheter, and methods of the present disclosure have advantageous use for gaining access to supra-aortic blood vessels by way of radial route in patients who have tortuous aortic arch and, in particular, advantageous as compared to using the trans-femoral route for accessing the supra-aortic vessels with a catheter.

In embodiments, systems, medical devices, catheters, methods of diagnosis, and methods of therapy, of the present disclosure are not constrained by the tortuosity of a given blood vessel, not constrained by the arch width, not constrained by curvature radius, and not constrained by attachment zone angles.

In embodiments, systems, medical devices, catheters, and methods of the present disclosure, are not constrained to cerebrovascular procedures that involve radial access.

In embodiments, systems, medical devices, catheters, and methods of the present disclosure, can be used for diagnosis or for treatment of cerebral strokes. Systems, medical devices, catheters, and methods of the present disclosure are not constrained to the indication of cerebral strokes, and are not constrained to patients with bovine arch variant, and are not constrained to patients with wound arch.

In embodiments, systems, medical devices, catheters, and methods of the present disclosure, can be used for diagnosis or for treatment of aberrant vessels that form vascular rings where these vascular rings compress the trachia or compress the esophagus (see, Sechtem, Fisher, Higgins (1987) AJR Am. J. Roentgenol. 149:9-13). Systems, medical devices, catheters, and methods of the present disclosure, can be used for diagnosis or for treatment of right aortic arch with mirror-image branching, and can be used via the left radial artery in such a scenario.

In exclusionary embodiments, systems, medical devices, catheters, and methods of the present disclosure, are not used for the diagnosis and are not used for the treatment of left aortic arch with aberrant right subclavian artery.

In embodiments, systems, medical devices, catheters, and methods of the present disclosure can be used for the diagnosis or for treatment of double arch. In embodiments, systems, medical devices, catheters, and methods of the present disclosure can be used for the diagnosis or for treatment of carotid artery stenosis (see, Saxena, Ng, Lim (2019) Imaging modalities to diagnose carotid artery stenosis. BioMedical Engineering. 18:66).

In embodiments, systems and methods of the present disclosure includes three dimensional magnetic resonance angiography, before, during, or after treating a patient (see, Krinsky, Rofsky, Weinreb (1996) Gadolinium-Enhanced Three-Dimensional MR Angiography of Acquired Arch Vessel Disease. AJR. 167:981-987). In embodiments, system and methods of the present disclosure includes computed tomography, before, during, or after treating a patient (see, Boufi, Loundou, Alimi (2017) Eur. J. Vase. Endovasc. Surg. 53:663-670). In embodiments, system and methods of the present disclosure includes magnetic resonance Imaging (MRI), before, during, or after treating a patient (see, Sechtem, Fisher, Higgins (1987) AJR Am. J. Roentgnol. 149:9-13).

Guidance for placing a medical device during use in a patient, can be provided, by ultrasound or optical coherence tomography (Muraoka et al (2012) 28:1635-1641; Kang et al (2011) Circ. Cardiovasc. Interv. 4:139-145; Alfonso et al (2012) 103:441-464). Medical device can be configured, for placing at or near neointimal formation, location at risk for restenosis, atherosclerotic plaque, bile tract, urinary tract, lymphatic duct, intestines, pulmonary tract, and so on. Identifying lesions at risk for restenosis can be made by available methods (see, e.g., Montalescot et al (1995) Circulation. 92:31-38; Killip et al (1995) J. Nuclear Med. 36:1553-1560; Garg et al (2008) J. Am. College Cardiol. 51:1844-1853).

In exclusionary embodiments, systems, catheters, medical devices, and methods of the present disclosure can exclude any system, medical device, catheter, or method that uses three dimensional magnetic resonance angiography, that uses magnetic resonance imaging (MRI), or that uses computed tomography.

The Cardiac Arch (Types and Variations)

Systems, medical devices, compositions, pharmaceutical agents, and the methods of the present disclosure can be used for treating, for diagnosing, or for treating and diagnosing, cardiovascular disorders. The conclusion of the diagnosis can be that a given organ or tissue is healthy, or it can result in the diagnosis that a given organ or tissue comprised a disorder. Relevant healthy conditions and relevant disorders include, “Young Arch,” “Bovine Arch,” and “Unwound (Aged) Arch.” These terms refer to the aortic arch. The Young Arch, which is often characterized as the most commonly occurring anatomy of the aortic arch, has been called Type I aortic arch (see, Rojas, Muete, Qijano (2017) Rev. Fac. Med. 65:49-54). A young arch is what we see in the younger population, say 45-50 years and younger. Bovine Arch has been called Type II aortic arch (see, page 133 of Moorehead, Miller, Kashyup (2016) Ann. Vasc. Surg. 30:132-137, Rojas, Muete, Qijano (2017) Rev. Fac. Med. 65:49-54). Unwound Arch has been called, Type III aortic arch (see, page 229 of Vascular Medicine and Endovascular Interventions (ed. by T. W. Rooke) Blackwell Futura (2007)).

Young Arch.

A young arch is what occurs in younger human populations, typically, 45 to 50 years old and younger. Three vessels branch directly from the upper-curved region of the aortic arch in normal people. These three vessels are: (1) INNOMINATE ARTERY (also called, Brachiocephalic trunk and Brachiocephalic artery); (2) LEFT COMMON CAROTID ARTERY; and (3) LEFT SUBCLAVIAN ARTERY. From these three vessels, additional vessels branch off, and these are as follows (see, below):

(1) BRANCHING FROM INNOMINATERTERY. There is a branching point, where the innominate aorta has a first branch (right carotid artery), and a bit further farther from the aortic arch is a second branching point (right vertebral artery), and beyond this second branch the innominate artery is called, right subclavian artery). See, Layton, Kallmes, Cloft (2006) AJNR Am. J. Neuroradiol. 27:1541-1542.

(2) LEFT CAROTID ARTERY. Left carotid artery does not have any further branches, at least not in the figure provided by, Layton, Kallmes, Cloft (2006) AJNR Am. J. Neuroradiol. 27:1541-1542.

(3) LEFT SUBCLAVIAN ARTERY. Left subcavian artery has at least one branch, the left carotid artery. See, Layton, Kallmes, Cloft (2006) AJNR Am. J. Neuroradiol. 27:1541-1542.

BOVINE ARCH.

Bovine arch is similar to normal configuration of human aortic arch, except that only two vessels (not three vessels) branch off directly from the upper-curved region of the aortic arch. Bovine arch has at least two variations, as described below. In each of these two variants, what is ordinarily the central (the left carotid artery) of three branches in normal people, now branches off from the trunk of innominate artery. In bovine arch, when the left carotid artery branches off from innominate artery, it is still called, “left carotid artery” See, Layton, Kallmes, Cloft (2006) AJNR Am. J. Neuroradiol. 27:1541-1542.

BOVINE ARCH (first variant). In this variant of bovine arch, the left carotid artery branches off from the trunk of the innominate artery. The branching point from innominate artery can begin immediately above the top surface of aortic arch. Described another way, the branching point from innominate artery occurs close to top surface of aortic arch, and far away from branching point of right carotid artery. See, Layton, Kallmes, Cloft (2006) AJNR Am. J. Neuroradiol. 27:1541-1542. Another group of researchers (Dumfarth, Plaikner, Krapf (2014) Ann. Thorac. Surg. 98:1339-1346) refers to this first variant as the, “more frequent variant.”

BOVINE ARCH (second variant). In this variant of bovine arch, the left carotid artery branches off from the trunk of the innominate artery. The branching point from innominate artery can begins at a point that is half-way between the top surface of aortic arch, and the branching point of right carotid artery. See, Layton, Kallmes, Cloft (2006) AJNR Am. J. Neuroradiol. 27:1541-1542. Another group of researchers (Dumfarth, Plaikner, Krapf (2014) Ann. Thorac. Surg. 98:1339-1346) refers to this first variant as the, “less frequent variant.”

UNWOUND AGED ARCH (USUALLY CALLED TYPE III ARCH). The term “unwound arch” is synonymous with a “Type III arch.” Type III arch is a condition found in elderly people. In this type of aortic arch, the origin of the innominate/brachiocephalic artery lies caudal (meaning, it lies below) the inner curve of the aortic arch. It has been proposed that unwound arch in aged people occurs because, “a lifetime of continual downward drag of a . . . pulsating heavier heart tends to gradually pull the rightward aspect of the aortic arch caudad” (see, Tayal, Barvalia, Wasty (2016) J. Cardiology & Current Research. Vol. 6 (5 pages)). With normal heart anatomy (Type I arch), the vertical distance between origin of brachiocephalic artery and top of arch is under one diameter of the left common carotid artery (LCCA). With Type II arch, this distance is between one and two LCAA diameters. But with Type III arch (the unwound arch), this distance is greater than two LCCA diameters (see, Tayal, Barvalia, Wasty (2016) J. Cardiology & Current Research. Vol. 6 (5 pages)). Regarding the aged arch, one source states that, “With increasing age, the aorta tends to unfold and elongate, with great vessels origin being displaced caudally. This creates a steeper aortic arch over time and spreads the origins of the great vessels as well as altering their angle of take-off relative to the top of the arch” (see, page 151 of Carotid Artery Stenting (2009) edited by Jacqueline Saw. Humana Press).

In embodiments, system, medical device, pharmaceutical agent, and methods of the present disclosure can encompass use with a patient that has one of the above types of aortic arch. Also, system, medical device, pharmaceutical agent of the present disclosure can encompass a medical device that is capable of use with a patient who has one of the above types of aortic arch. Said system, medical device, or pharmaceutical agent can be capable of use with a population of patients that have only one, only two, only three, only four, or more, of the above types of aortic arch.

In exclusionary embodiments, system, medical device, pharmaceutical agent, and methods of the present disclosure, can exclude any system, device, medical device, pharmaceutical agent, and method, that comprises one or more of the above types of aortic arch, or that comprises interactions with one or more of the above types of aortic arch, or that comprises diagnostic use or treatment use with a patient that comprises one of the above types of aortic arch.

Steeper aortic arch produces difficulty in gaining access to target vessel. Where the physicians goal is carotid intervention the steeper the arch and the more inferior the origin of the arch, especially when the innominate artery is accessed by way of the femoral route, the result of the steeper arch and the more inferior origin, is greater difficulty in accessing the target vessel (see, page 152 of Carotid Artery Stenting (2009) edited by Jacqueline Saw. Humana Press).

Aortic Arch Classification by Angles and, Alternatively, Aortic Arch Classification (Types I, II, and III) Based on Vertical Distance Between Origin of Bracihocephalic Artery (Innominate Artery) and Top of Arch.

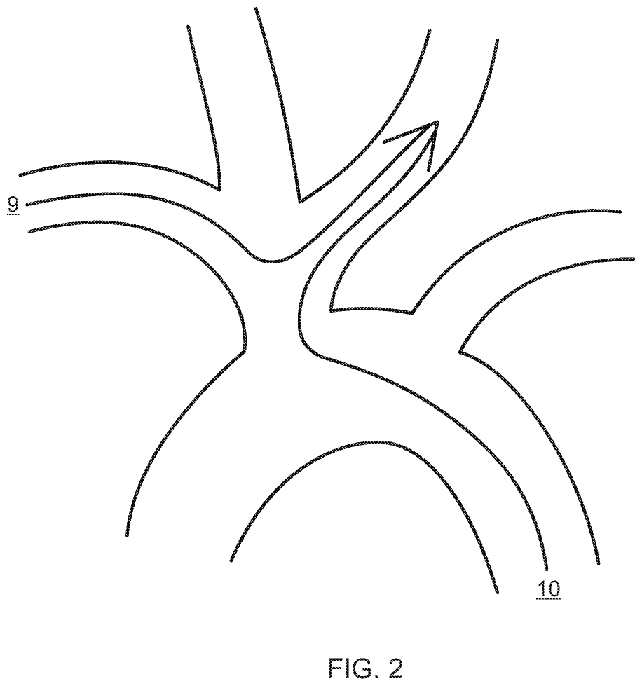

Two different classification systems are described by Demertzis and Stalder (2010) Journal of Anatomy. 217:588-596. The Demertzis reference discloses that the aortic arch can be described in three ways: (i) By the angle between a line connecting the highest point of the aortic arch and a mid-luminar point of the ascending and descending aorta at the height of the bifurcation of the pulmonary trunk in the parasagittal plane (FIG. 2D in Demertzis), (ii) By the angle between the horizontal plane and the long axis of the three arch segments defined by the origins of the supra-aortic arteries in the axial view (see, curvatures 1, 2, and 3, in FIG. 2 of Demertzis), and (iii) Described as Type I, Type II, or Type III, using the criterion of vertical distance from the top origin of the brachiocephalic trunk to the top of the arch in the parasagittal stretched-out projection. This distance is less than one diameter of the left common carotid artery in a Type I arch, between one and two diameters in a Type II arch, and greater than two diameters in a Type II arch (see, page 589-560 in Demertzis). This same Type I, Type II, and Type III classification system is also described by, Tayal, Barvalia, Wasty (2016) J. Cardiology & Current Research. Vol. 6 (5 pages)).

In embodiments, system, medical device, catheter, methods of diagnosis, and methods of treatment of the present disclosure can be used with a patient with one of the above types of aortic arches (as disclosed by angle classification or as disclosed by Type I, Type II, and Type III classification), or can be used with a patient population with two of the above types, with three of the above types, with four of the above types, with five of the above types, or with all six of the above types. In exclusionary embodiments, system, medical device, catheter, methods of diagnosis, and methods of treatment of the present disclosure can exclude use with patients with one of the above types of aortic arches (as disclosed by angle classification or as disclosed by Type I, Type II, and Type III classification), or can be used with a patient population with two of the above types, with three of the above types, with four of the above types, with five of the above types, or with all six of the above types.

Type I, Type II, Type III classification based on angle versus Type I, Type II, Type III classification that distinguishes between young arch, bovine arch, and unwound arch. The terms, “Type I,” “Type II,” and “Type III” have been used for three distinct classification systems. The system based on angle is disclosed above, in the account of the Demertzis reference and the Tayal reference. The second system is where the term, “Type II,” is used to refer to bovine arch (see,

Moreover, within the Type II (bovine arch), there exist subtypes, where each subtypes is a variant of the bovine arch (see, Hornwick, Mojibian, Rizzo (2012) Cardiology. 123:116-124; Moorehead, Kashyup, Kendrick (2016) Ann. Vasc. Surg. 30:132-127: Spacek and Veselka (2012) Bovine Arch, Letter to Editor. Arch. Med. Sci. 8:166-167).

Yet another classification of aortic arch variants is available (see, Popieluszko, Henry, Sanna (2018) J. Vascular Surgery. 68:298-306). Type 1, normal: The AA went from right to left, giving off these branches: BT, LCC, and LS arteries. Type 2, bovine arch: The AA went from right to left, giving off these brances: a common trunk giving rise to the BT and the LCC, followed by the LC. Type 3. LV: The AA went from right to left, giving off the BT, LCC, LV, and LS arteries. Type 4, bovine and LV: The AA went from right to left and gave off a common trunk of the BT and the LCC, followed by the LV and LS. Type 5. common carotid: The AA went from right to left, giving off the RS artery, followed by a common trunk for the RCC and LCC and the LS. Type 6. aberrant RS: The AA went from right to left, giving off an RCC and LCC, an LS, and an aberrant RS. Type 7. right arch: The AA went from left to right, giving a mirrored pattern or an aberrant LS (see, pages 1 and 3 of Popieluszko, Henry, Sanna (2018). Vascular Surgery. 68:298-306). LCC is common carotid artery. LS is left subclavian artery. RCC is right common carotid artery. RS is right subclavian artery. BT is brachiocephalic trunk.

In embodiments, system, medical device, pharmaceutical agent, and methods of the present disclosure can encompass use with a patient that has one of the above types of aortic arch. Also, system, medical device, pharmaceutical agent of the present disclosure can encompass a medical device that is capable of use with a patient who has one of the above types of aortic arch. Said system, medical device, or pharmaceutical agent can be capable of use with a population of patients that have only one, only two, only three, only four, or more, of the above types of aortic arch.

In exclusionary embodiments, system, medical device, pharmaceutical agent, and methods of the present disclosure, can exclude any system, device, medical device, pharmaceutical agent, and method, that comprises one or more of the above types of aortic arch, or that comprises interactions with one or more of the above types of aortic arch, or that comprises diagnostic use or treatment use with a patient that comprises one of the above types of aortic arch.

Influence of Aortic Arch Variants on the Ability to Treat Various Cardiovascular Disorders

Innominate Artery Compression Syndrome (IACS). “Innominate artery” is also called, “brachiocephalic artery” (see, page 128 of Grant, Dempsey, Harrison (2006) Brit. J. Anaesthesia. 96:127-131). Similarly, “innominate artery” is sometimes called, “brachiocephalic trunk” (see, title of Fawcett, Gomez, Hughes (2010) Clinical Anatomy. 23:61-69). Brachiocephalic trunk of the aortic arch crosses the trachea, and can compress the trachea. This compression can result in Innominate Artery Compression Syndrome (IACS), where the branching point of the innominate artery is, “somewhat to the left of its normally expected position” on the aortic arch. IACS occurs in children, and symptoms of IACS may or may not spontaneous decrease as the child matures (Fawcett, Gomez, Hughes (2010) Clinical Anatomy. 23:61-69).

ANATOMICAL VARIATIONS IN AORTIC ARCH AND CALCIFICATION CAN IMPAIR CATHETERIZATION. As people age, the aortic arch becomes calcified, elongated, and less compliant. As the arch elongates, it extends beyond the origin of the left subelavian artery, which, along with other supra-aortic great vessels, tethers the aortic ostial segment. This results in much greater difficulty in catheterization of these great vessels from a transfemoral approach. “Anatomical variations in the aortic arch, such as the . . . bovine arch, can complicate access to the great vessels . . . these arches also . . . have atherosclerotic plaque . . . posing . . . higher risk of intraprocedural embolic complications” (see, Shakir, Siddiqui (2017) Neurosurgical Focus. 42:E14 (5 pages)). In a small number of patients, the tortuosity of the arch and elongation and tortuosity of the great vessels impair attempts with standard glide wires (or guide wires) through recurve catheters result in a catheter “kickback” into the aorta and failure to achieve access (see, Shakir, Siddiqui (2017) Neurosurgical Focus. 42:E14 (5 pages)).

In addition to impairing access by catheters, variations in the aortic arch, such as bovine arch (Type II aortic arch), can “increase risk for complications during surgical procedures.” For example, bovine arch can make, “carotid stenting more difficult and risky . . . [where] this difficulty may be encountered as a result of a tight turn involving the brachiocephalic trunk (BT) and the common carotid artery (LCC) arteries on stending through a femoral approach.” (see, Popieluszko, Henry. Sanna (2017) Journal Vascular Surgery. 68:298-306).

Drugs that Prevent or Reduce Vasospasms.

The present disclosure provides agents that prevent vasospasm or that reduce an existing vasospasm, including verapamil, verapamil with nitroglycerin, nitroglycerin, clonidine, milrinone, and isosorbide dinitrate.

Guidance on administrating verapamil and other agents that prevent or reduce vasospasm is available. For example, intraarterial verapamil can be administered proximally in the affected vascular territory. Verapamil can be diluted in saline to a final concentration of mg/mL and pulse-infused at a rate of 1 mL/min through a catheter, meaning that 1 mL of verapamil was injected every minute through the side port of a 3-way valve connected to the catheter (see, Ospel Jung, Vidal (2020) Am. J. Neuroradiol. 41:293-299).

Nitroglycerin or a nitrate can relieve vasospasm by being converted into nitric oxide (NO), where this conversion occurs inside the patient, and where this nitric oxide (NO) then stimulates guanylate cyclase, resulting in increased cyclic GMP and consequent vasodilation (Yasue and Kugiyama (1997) Internal Medicine. 36:760-765).

In exclusionary embodiments, system, medical devices, instruments, compositions, pharmaceutical agents, and methods of the present disclosure can exclude any one or more of the above agents that prevent vasospasms or that reduce vasospasms.

Systems, devices, coatings, slurries, suspensions of the present disclosure encompass nanospheres, where the nanospheres contain one or more drugs, and where the drugs slowly elute from the nanospheres. Nanospheres and other types of nanoparticles for delivering drugs have been made from the following materials. ZIF-8 is a biocompatible metal organic framework (see, Zhuang, Kuo, Weerapana (2014) ACS Nano. 3:2812-2819). Mesoporous silica nanospheres are available, with or without functionalization with hydroxyl groups or with carboxyl groups. Nanospheres can be made from, PLGA (poly(D,L-lactide-co-glycolide) (see, Tafaghodi, Jaafari (2011) Parasitol. Res. 108:1265; Deepika, Thangam (2019) Materials Science Engineering:C. vol. 103). Nanospheres made with polycaprolactone (PCL), Eudragit® RS100 copolymer, polycaprolactone (PCL), or Purac® (Corbion, Lenexa, Kans.; Biomaterials, Netherlands) (see, Yousry and Elshafeey (2018) J. Drug Delivery. 25:1448-1460).

Reagents and Equipment

Pharmaceutical agents, drugs, and other chemical reagents, and laboratory equipment, are available (see, e.g., Sigma Aldrich, St. Louis, Mo., Thermo Fisher Scientific. Waltham, Mass.). Surgical supplies, tools, and equipment, are available (see, e.g., Stryker, Kalamazoo, Mich., DiaMedical, West Bloomfield, Mich., Medtronic, Fridley, Minn.). Components for the methods and devices of the disclosure are available, for example, from Advanced Cardiovascular Systems in Santa Clara, Calif.; Baxter International of Deerfield, Ill.; Abbott Laboratories at Abbott Park, Ill., Edwards Lifesciences, Irvine, Calif., and Boston Scientific of Natick, Mass. Components of the present disclosure can be made, without limitation, by molding, blow molding, slush molding, injection molding, rotational molding, compression molding, extrusion, thermoforming, stamping, calendaring, and so on (Brazel, CS; Rosen, S L (2012) Fundamental Principles of Polymeric Materials. Wiley, Hoboken, N.J.).

A composition that is “labeled” is detectable, either directly or indirectly, by spectroscopic, photochemical, biochemical, immunochemical, isotopic, or chemical methods. For example, useful labels include ³²P, ³³P, ³⁵S, ¹⁴C, ³H, ¹²⁵I, stable isotopes, epitope tags fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

In exclusionary embodiments, the present disclosure can exclude any system, device, instrument, reservoir, coating, or method that contains one or more of the above agents.

Polymers for Making Medical Devices

Thermoplastic polyurethane (TPL) tubing, resins, and the like, are available for use in the present disclosure, for example, as a medical device such as a catheter, as a coating for the medical device, as a formula configured for use in coating the medical device, or as a medical device that is modified by coating with the formula. What is available is tubing, resins, and the like, having a hardness of 72 A, 77 A, 87 A, 94 A, 51 D, 60 D, 63 D, 67 D, 73 A/78 A, 83 A/86 A, 90 A/95 A, 93 A/98 A, 55 D/65 D, 63 D/78 D, 73 D, 75 D/82 D (Tecofex® series); and 75 A, 85 A, 94 A, 54 D, 64 D, 69 D, 74 D, 75 D, 77 A/83 A, 87 A/88 A, 97 A/97 A, 55 D/64 D, 67 D/75 D, 70 D, 75 D, 77 D/84 D (Tecothane® series) (Lubrizol's Engineered Polymers for Medical and Health Care; Lubrizol Corp, Cleveland Ohio). Guidance on medical polymers, including polyurethane, is available, for example, from Polymer Membranes/Biomembranes (Advances in Polymer Science), ed. by Meier and Knoll, Springer, 2009; Lubricating Polymer Surfaces by Uyama, CRC Press, 1998; and Polymer Grafting and Crosslinking, ed. by Bhattacharya, et al, Wiley, 2008.

Polyurethane copolymers, polyurethane polymers, and polyurethane block polymers for use in manufacturing medical devices are available from DSM Biomedical, Exton, Pa. (see, U.S. Pat. No. 9,393,311 of Ward, which is incorporated herein by reference in its entirety).

Hydrophilic polymer can consist of or, alternatively, can comprise, poly(lactams), for example polyvinylpyrollidone (PVP), polyurethanes, homo- and copolymers of acrylic and methacrylic acid, polyvinyl alcohol, polyvinylethers, maleic anhydride based copolymers, polyesters, vinylamines, polyethyleneimines, polyethyleneoxides, poly(carboxylic acids), polyamides, polyanhydrides, polyphosphazenes, cellulosics, for example methyl cellulose, carboxymethyl cellulose, hydroxymethyl cellulose, and hydroxypropylcellulose, heparin, dextran, polysacharrides, for example chitosan, hyaluronic acid, alginates, gelatin, and chitin, polyesters, for example polylactides, polyglycolides, and polycaprolactones.

Also, hydrophilic polymer coating can comprise poly(actams), for example polyvinylpyrollidone (PVP), polyurethanes, homo- and copolymers of acrylic and methacrylic acid, polyvinyl alcohol, polyvinylethers, maleic anhydride based copolymers, polyesters, vinylamines, polyethyleneimines or polyethyleneoxides.

Hydrophilic polymer can be wetted by (or can comprise) one or more of, diethyeneglycol, triethyleneglycol, tetraethyleneglycol, propyleneglycol, dipropyleneglycol, triprolyeneglycol, ethanolamine, diethanolamine, triethanolamine, and polyethylene glycol.

Copolymers are encompassed by the disclosure, for example, copolymers of the block type and copolymers of the rake type (see, e.g., U.S. Pat. No. 8,008,407 of Oberhellman et al, and U.S. Pat. No. 8,084,535 of Maton et al, which are incorporated herein by reference in their entirety). Regarding porosity, if the porosity of a polymer coating is not sufficient to allow diffusion of an agent, such as a drug, into the extracellular fluids, a porosigen, such as lactose, can be added to the polymer used for the coating. Hydrogels, and methods for controlling water content of hydrogels, and mechanical strengths of various types of hydrogels are described (see, e.g., U.S. Pat. No. 4,734,097 of Tanabe et al, which is hereby incorporated by reference in its entirety). Because of their weak, rubbery mechanical properties, polysiloxane is sometimes prepared as chemically crosslinked, or synthesized as a block polymer that alternates with a harder type of polymer (see, page 36 of F. Wang (1998) Polydimethylsiloxane Modification of Segmented Thermoplastic Polyurethanes and Polyureas, Thesis, Virginia Polytechnic Institute and State Univ., Blacksburg, Va.).

An example of “one type” of plastic polymer is, for example, a polymer that comprises mainly polyurethane, mainly polysiloxane, mainly polyethylene, or mainly one type of copolymer. The skilled artisan will understand that modification of a polyurethane polymer with various end groups do not change the fact that the polymer is still classified as a type of “polyurethane.” A “copolymer” is defined as consisting mainly of “one type” of plastic polymer, because the two polymers in the copolymer are integrated together, and are also covalently bound to each other, for example, in the manner of a block copolymer or a rake copolymer. The term “mainly” can mean, without limitation, at least 99% by weight of a given polymer (excluding any solvents or solutions that might be associated with or entrapped in the polymer), at least 98%, at least 95%, at least 90%, at least 85%, at least 80%, at least 75%, at least 70%, and so on.

French Size

Diameters of catheters, cannulas, tubes, and such, can be labeled by French size. The present disclosure provides a tube with a French size that is, to provide non-limiting examples, 3 Fr (1 mm; 0.039 inches), 4 Fr (1.35 mm; 0.053 inches), 5 Fr (1.67 mm; 0.066 inches), 6 Fr (2 mm; 0.079 inches), 7 Fr (2.3 mm; 0.092 inches), and so on. The corresponding diameters in millimeters and inches are shown in parenthesis. The French system has uniform increments between gauge sizes (⅓ of a millimeter) (Iserson K V (1987) J.-F.-B. Charriere: the man behind the “French” gauge. J. Emerg. Med. 5:545-548). Systems for measuring the outside diameter and inside diameter (lumen) of catheters, needles, and the like have been described (see, e.g., Ahn, et al. (2002) Anesth. Analg. 95:1125). French size can refer to an inside diameter or to an outside diameter (see, e.g., U.S. Pat. No. 7,641,645 issued to Schur, which is hereby incorporated by reference in its entirety).

In exclusionary embodiments, system, medical device, catheter, and methods of the present disclosure can exclude any system, medical device, catheter, sheath, and related methods, that comprise a catheter or a sheath that meets one or more of the above French values.

Coatings

The present disclosure provides, without limitation, coatings that comprise sulfobetaine, or carboxybetaine, hydrogels, polyurethane, polyester, polyethylene, polyamide, mixtures thereof, diblock polymers, layered coatings, interpenetrating polymer networks, See, e.g., U.S. Pat. No. 7,879,444 issued to Jiang et al; US 2009/0259015 of Jiang and Chen; US 2009/0155335 of O'Shaughnessey et al; US 2009/0156460 of Jiang et al; US 2010/0145286 of Zhang et al; 2011/0097277 of Jiang et al; and US 2010/0152708 of Li et al, each of which is individually incorporated herein by reference in its entirety.

What is embraced is a formulation for applying to a surface of a medical device, for example, by soaking, where the formulation comprises a dissolved plastic polymer. The dissolved plastic polymer can be more or more of, or any combination of, polyurethane, polyethylene, polyethlyene teraphthalate, ethylene vinyl acetate, silicone, tetrafluoroethylene, polypropylene, polyethylene oxide, polyacrylate, and so on. What is encompassed are coatings, coating solutions, and medical devices that are coated with coating solutions, using Carbothane® family of polycarbonate-based aliphatic and aromatic polyurethanes, Estane®, which is a thermoplastic polyurethane, Pellethane®, which is a family of medical-grade polyurethane elastomers and exceptionally smooth surfaces, Tecoflex®, which is a family of aliphatic polyether polyurethanes, where low durometer versions are particularly suitable for long-term implant applications, Tecothane®, an aromatic polyurethane, Texin®, an aromatic polyether-based polyurethane which allows for very thin gauges (Microspec Corp., Peterborough, N.H.; Lubrizol, Inc., Wickliffe, Ohio; Entec Polymers, Orlando, Fla.). See, U.S. Pat. No. 6,565,591 of Brady, U.S. Pat. No. 7,029,467 of Currier, and U.S. Pat. No. 7,892,469 of Lim, which are hereby incorporated by reference in their entirety. In embodiments, the present disclosure provides the recited polymers for use in coating solutions, or for use in manufacturing the medical device that is to be coated. A reagent, such as an anti-microbial agent, can be bulk distributed in the medical device, for example, by adding to a melted polymer or by soaking until even distribution has occurred.

Coating can be via spray-coating, dip-coating, brushing, or vacuum-deposition.

Coating can be with a polyurethane/tetrahydrofuran solution to create a polyurethane matrix, where coating solution includes, or does not include, an anti-vasospasm drug. Coating can be with a solution that is capable of providing timed-release of the anti-vasospasm drug from the catheter's coating. In an exclusionary embodiment, system, medical devices, catheters, and methods of the present disclosure can exclude any system, medical device, catheter, or method, where the coating includes any drug that is other than an anti-vasospasm drug. Also, what can be excluded is any medical device that comprises an anti-restinosis agent or drug, or that comprises an anti-inflammatory agent or drug, or that comprises an anti-proliferative drug, or that comprises an anti-cancer drug, or that comprises an anti-neoplastic drug.

Coating can be a polymer, where the polymer is comprised of one or more different types of monomers, where the monomer can be, 2-ethoxyethyl methacrylate, an acrylate monomer, hexyl methacrylate, butyl methacrylate, ethyl methacrylate, lauryl methacrylate, hydroxyl propylmethacrylate, hydroxyl ethylmethacrylate, methyl methacrylate, or ethylacrylate. In exclusionary embodiments, the system, medical device, catheter, and methods of the present disclosure can exclude any medical device that comprises one or more of the above monomers or that comprises a polymer that comprises one or more of the above monomers.

In exclusionary embodiments, system, device, instruments, medical device, coating, device made of or with plastic, and methods of the present disclosure can exclude any medical device, any part, any structure, any coating, that includes one or more of the above molecules or that includes one or more of the above polymers.

Alternatively, the medical device can be impregnated or coated with the agent. In embodiments, the disclosure encompasses methods for bulk distribution, gradient distribution, and limited surface distribution. Methods for manufacturing medical devices where an agent is bulk distributed, gradient distributed, or limited surface distributed, are available (see, e.g., U.S. Pat. No. 4,925,668 issued to Khan, et al, U.S. Pat. No. 5,165,952 issued to Solomon and Byron, and U.S. Pat. No. 5,707,366 issued to Solomon and Byron, all of which are incorporated herein by reference).

Coating and impregnation are distinguished. Generally, coating resides on, or adheres to, the exterior surface of medical device. Coating thickness can be, without limitation, about 10 nanometers (nm), about 50 nm, about 100 nm, about 500 nm, about 1.0 micrometers (um), about 10 um, about 50 um, about 100 um, about 500 um, about 1 millimeters (mm), about 5 mm, and so on. Material used for coating can extend into the medical device, and this aspect of the coating can be referred to as an impregnation. Impregnation can extend throughout entire medical device, and where extension throughout device is substantially uniform, the impregnation is a bulk distribution. Impregnation can extend, without limitation, about 10 nanometers (nm), about 50 nm, about 100 nm, about 500 nm, about 1.0 micrometers (um), about 10 um, about 50 um, about 100 um, about 500 um, about 1 millimeters (mm), about 5 mm, and so on, from the surface into medical device. Alternatively, device can be manufactured so that an agent does not reside on the surface, but resides only in interior of medical device. Use of the term “coating” or “impregnation” can depend on whether the coating or the impregnation is functionally more important.

Manufacturing Coated Medical Devices

Medical devices of the present disclosure can be coated with, for example, a lubricious polymer using a machine, where available machines include RDX-XL Coating System, PCX Coating System, GWX Coating System, and 1DX Coating System (available from Harland Medical Systems, Eden Prairie, Minn.). These machines accomplish both dip-coating and curing, for example, curing with ultraviolet light or with heat.

Testing Friction of Coated Medical Devices

Machines are available for measuring lubricious coating performance on medical devices, such as on catheters or on guidewires. FTS 6000 Friction Test System) measures both surface friction and coating durability (available from Harland Medical Systems, Eden Prairie, Minn.). In embodiments of the present disclosure, the measured coefficient of friction can be lower than 12, lower than 8, lower than 6, lower than 4, lower than 2, lower than 1, lower than 0.8, lower than 0.6, lower than 0.4, lower than 0.2, lower than 0.1, lower than 0.05, and so on. In exclusionary embodiments, the present disclosure can exclude any system, device, catheter, and method, where the coefficient of friction does not meet one of the above ranges or does not meet one of the above cut-off points. The above machine conducts the standard “pinch test.” To run this test, the user fastens the sample in FTS sample holders, selects a test protocol, and then moves the transport to the desired starting position. Coefficient of friction (COF) is used to rate the performance of the coating. The COF is the measured friction\clamp force.

Coefficient of friction can be measured (see, e.g., Malkin and Harrison (1980) A small mobile apparatus for measuring the coeficient of friction of floors in J. Phys. D: Appl. Phys. 13 L77; Jay, et al (2007) Association between friction and wear in diarthrodial joints lacking lubricants in Arthritis Rheumatism. 56:3662-3669; Savescu, et al (2008) A technique to determine friction at the finger tips in J. Appl. Biomech. 24:43-50).

Wetting Agents for the Coating

The hydrophilic polymer coating on the medical device can be wetted by the wetting agent. In particular the term is used herein to describe a coating that contains-sufficient wetting agent to be lubricious. A lubricious coating can have a Coefficient of Friction lower than 0.15. Usually a wetted coating contains at least 10 wt. % of wetting agent, based on the dry weight of the coating, at least 50 wt. %, based on the dry weight of the coating, at least 100 wt. % based on the dry weight of the coating, or at least 300-500 wt. %, and the like.

Proximal and Distal

In the context of a medical device, such as an assembly having a longitudinal aspect, as an assembly of a sheath and dilator, “proximal” refers generally to the end of a catheter and sheath assembly that is closest to the physician while “distal” refers generally to the end that is initially inserted into the patient during a procedure where the entire catheter is intended to be inserted. Where the terms “proximal-to-distal movement” or “proximal-to-distal force” are used, these terms can refer to the context where the device is being used with the patient, and also in an abstract context, where a physician and patient are not present.

### Examples

Examples are disclosed by the text and the figures. Examples include the inventive catheter, variations thereof, and related methods for medical use and methods for manufacturing. Examples include a sheath, a board base, a wrist pad, a grip bar, and a table top.

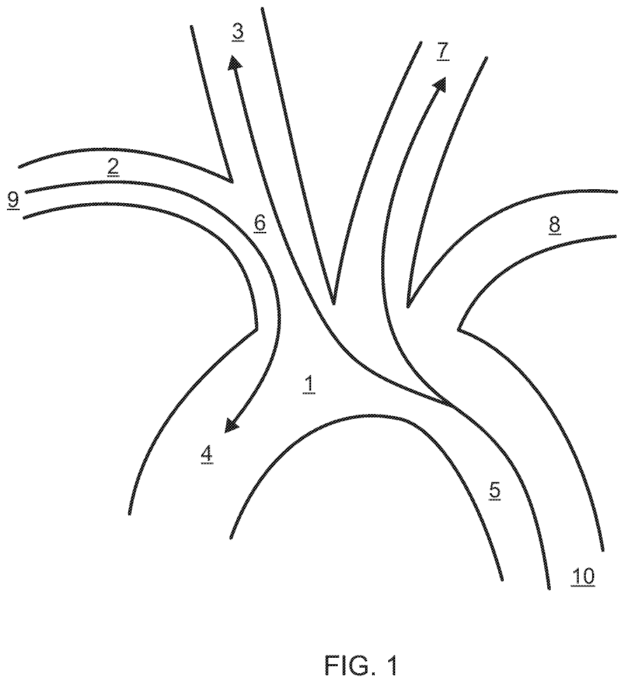

FIG. 1. YOUNG ARCH. (1) Aortic arch; (2) Right subclavian artery; (3) Right common carotid artery; (4) Ascending aorta; (5) Descending aorta; (6) Brachiocephalic trunk (also known as, brachiocephalic artery, and as innominate artery); (7) Left common carotid artery; (8) Left subclavian artery; (9) Radial access; (10) Femoral access.

FIG. 2. BOVINE ARCH. (9) Radial access; (10) Femoral access. Names of vessels are shown in the legged for FIG. 1.

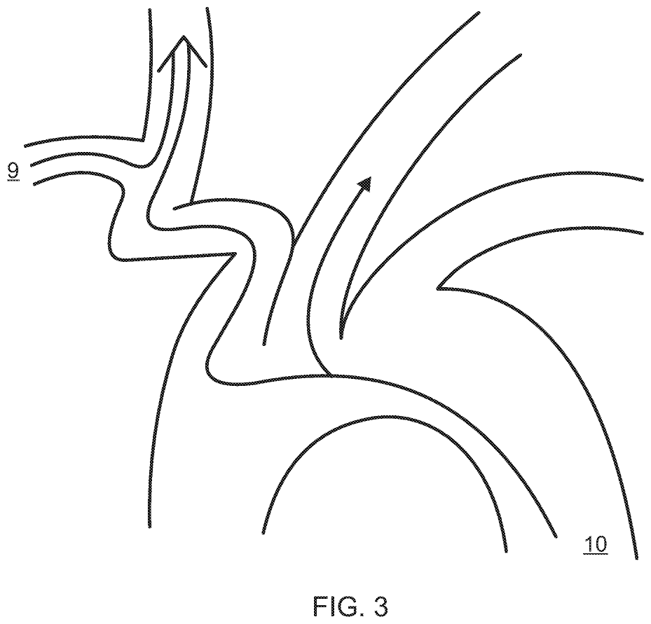

FIG. 3. UNWOUND (AGED) ARCH. (9) Radial access; (10) Femoral access. Names of vessels are shown in the legged for FIG. 1.

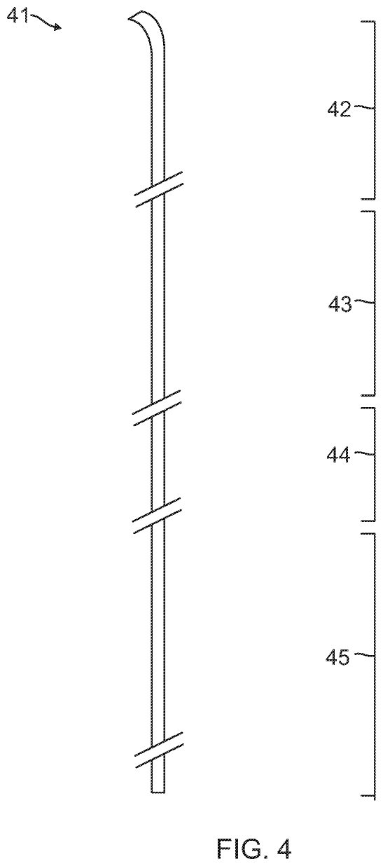

FIG. 4 discloses guidewire. 0.012 to 0.018 inch diameter tip (41). This tip is a preshaped tip or a shapeable tip. Preshaped tip is a tip that assumes a non-linear shape, such as a curved shape, when no exterior force is applied to tip. A shapeable tip can be a preshaped tip or a tip that is not pre-shaped, but where shape can be altered by force from physician's finger or by force encountered during passage through patient's vasculature.

Segment (42) of the guidewire is about 20 cm long. Segment (43) is taper segment, about 20 cm long, from 0.012 to 0.035 inches in diameter. Segment (44) is taper region to stiff region, about 10 cm long. Segment (45) is the stiff “arch” segment, which is about 150 cm long. Microwire segment (about 20 centimeters long) of the guidewire is indicatable by structure number (42).

CATHETER EMBODIMENTS (FIG. 5, FIG. 6, and FIG. 7).

FIG. 5 discloses a catheter that is the basic design, while FIG. 6 catheter includes S-shaped region, and FIG. 7 catheter includes flipped-tip.

Alternative Taper Embodiments for Each Version of Catheter.

Alternative Design catheter embodiments are configured for diagnostic angiography. Alternative designs for each of these three designs exist, where each of these alternative designs has a longer taper region, and where this longer taper region is preferably six centimeters long. In the alternative designs of FIG. 5 (basic design) and FIG. 7 (S-shaped region) the straight segment that immediately follows the tapered segment (in the non-alternative design) is deleted.

Starting from the proximal end of catheter in FIG. 6, what is encountered along the catheter is an arc that curves away from the hemisphere arc, which is followed by another arc, where this are curves towards the hemisphere arc. The catheter of FIG. 7 is more complicated than the catheter of FIG. 5, because near the distal end of the catheter of FIG. 7, the tube assumes a small semicircle.

Figure Five (Basic Design Catheter).

Proximal end is (51) (FIG. 5). Hub is indicated by structure (52). An alternate embodiment of basic design catheter does not have any hub. Basic design catheter has a first straight segment that is situated between the hub and structure number (53). Basic design has tapered straight segment, where the taper is from 6 French (wider) to 4 French (narrower), where this tapered straight segment resides between structure numbers (53) and (60). Basic design catheter has a second straight segment, that is three centimeters long, and that is defined as between structure numbers (60) and (54). Continuing on towards the distal terminus of the basic design catheter, is a first curved segment (three centimeter long, 30 degrees arc), and this is followed by semicircular segment (0.75 centimeter radius, 1.5 centimeter diameter), that terminates at an aperture having a 4 French outer diameter, and 0.038 inch inner diameter.

Alternative design introducing catheter specifications are, about 4 French entire length, and sheath specifications of 4 French internal diameter (ID) and 5 French outer diameter (OD).

Sheath embodiment of basic design catheter, where catheter and sheath can be assembled together. 6 French long sheath, with total length of 110 cm or 100 cm. 6 French (0.087 inch) minimal inner diameter and 7 French (0.099 inch) maximal outer diameter. Alternative design introducing catheter specifications are, about 4 French entire length, and sheath specifications of 4 French internal diameter (ID) and 5 French outer diameter (OD).

ALTERNATIVE DESIGN OF BASIC CATHETER. Alternative Design of basic catheter does not include the three centimeter tapered straight segment or the three centimeter straight segment, and where these two segments are replaced with a six centimeter tapered straight segment.

Inclusionary and Exclusionary Dimensions of Catheters of the Present Disclosure.

Length of any given segment of catheter, as measured along axis of interior lumen, can be 0.5 cm, 1.0 cm, 1.5 cm, 2.0 cm, 4.0 cm, 6.0 cm, 8.0 cm, 10 cm, 15 cm, 20 cm, 25 cm, 30 cm, 35 cm, 40 cm, 50 cm, 60 cm, 70 cm, 80 cm, 90 cm, 100 cm, 110 cm, 120 cm, 130 cm, 140 cm, 150 cm, 160 cm, 170 cm, 180 cm, 180 cm, 190 cm, 195 cm, 200 cm, 205 cm, 210 cm, 215 cm, 220 cm, and so on. Also, length of any given segment of catheter can be selected from any one of the above values, plus or minus two percent of that value, or plus or minus four percent of that value, or plus or minus six percent of that value, or plus or minus eight percent of that value.

In total length embodiments, the total catheter length as measured along axis of interior lumen can be selected from one of the above values, or from a range consisting of any two of the above values.

In exclusionary embodiments, system, medical device, and catheter of the present invention can exclude any medical device or exclude any catheter, where a segment of the medical device or catheter is greater than one or more of the above values, or where a segment of the medical device or catheter is lesser than one or more of the above values.

Figure Six (Catheter with S-Shaped Region).

FIG. 6 discloses catheter with a curved S-shaped region, where S-shaped region is situated between the distal semicircle and the three-centimeter long tapered segment. The S-shaped region consists of two segments, where the central point of each of these two segments are relatively far away from each other. Central point is defined elsewhere in this disclosure, and is used to define relative orientation of any two curved segments. In short, if central points of two curved segments are near each other, the two curved segments likely curve in the same general direction, but if central points of two curved segments are relatively far from each other, the two curved segments likely curve in opposite directions.

Descriptions of Orientations of the Two Segments of S-Shaped Region Relative to the Semicircular Segment Located at Catheter's Distal End.

Starting from the proximal end of S-shaped region (FIG. 6), the catheter first curves away from the semicircle and, in traveling from proximal-to-distal direction, S-shaped region then curves back towards the semicircle. The curve away from the semicircle is 30 degrees (a first three cm segment), and the curve towards the semicircle is 30 degrees (a second three cm segment). Distance between (57) and (58) is diameter of distal semicircle, and this distance is 1.5 cm (FIG. 6). Distance from (56) to (57) is 0.5 centimeters. Actually, the segment that is identified as an arc that is cut from a circle, is actually cut from a circle that is slightly oval, where this slightly oval configuration is indicated by the fact that diameter of semicircle is 1.5 centimeters and radius (the depth of the semicircle) is 0.5 centimeters, instead of being a 0.75 centimeter radius.

Some of the structure numbers for catheter of FIG. 6 are the same as structure numbers of FIG. 5, because they designate the same type of part that exists in each of these two catheters, even though not all of the parts in the FIG. 6 catheter are found in the FIG. 5 catheter. FIG. 6 discloses, proximal end (51), hub (52), straight segment that begins immediately distal to hub and continues to structure number (53)), tapered segment from (53) to (54) that is three centimeters long, where taper begins at 6 French at (53) and concludes at 4 French at (54).

First curved region in S-shaped curve (FIG. 6). Curve between (54) and (63) is a 30 degree curve, and where distance along this curved region is 3 cm. Second curved region in S-shaped curve. Curve between (63) and (55) has distance along curved region of 3 centimeters, and where this curve ahs 30 degree curve. Distance (61) is distal catheter length, which is 11.2 centimeters. Distance (62) is proximal catheter length, which is 103.8 cm or 113.8 cm. Total catheter length is 115 cm or 125 cm (FIG. 6).

Sheath embodiment that can be used with catheter of FIG. 6, is a sheath that is 110 cm or 100 cm long. Sheath is 6 French (0.087 inches min) inner diameter (ID) and 7 French (0.699 inch max) outer diameter (OD)). Introducing catheter specifications are, about 4 French entire length, and long sheath specifications of 4 French internal diameter (ID) and 5 French outer diameter (OD).

ALTERNATIVE DESIGN OF CATHETER WITH S-CURVE (FIG. 6). Alternative design of catheter with S-curve does not have the three centimeter tapered straight segment (tapered from 6 French down to 4 French), and where this three centimeter tapered segment is replaced with a six centimeter tapered straight segment (tapered from 6 French down to 4 French).

Figure Seven (Flipped-Tip Catheter).

FIG. 7 shows a catheter with flipped-tip, where the distal end has a semicircle, and where distal to this semicircle is the flipped-tip, and where this flipped tip consists of a 0.5 centimeter straight segment and a one centimeter long curved segment that assumes a 45 degree arc, where this are curves away from the semicircle.

Catheter of FIG. 7 does not include the S-shaped region that is part of catheter of FIG. 6, but catheter of FIG. 7 does have an additional curved region, as compared with the catheter of FIG. 5. This additional curved region is part of the flipped-tip.

FIG. 7 discloses, proximal end (51), hub (52), straight region that extends from distal end of hub (52) to structure number (53), two centimeter-long straight tapered segment from (53) to (54) where taper begins at 6 French and concludes at 4 French, followed in continuing from proximal-to-distal direction, with a two centimeter long straight segment, which is then followed with a six centimeter-long curved segment having a 45 degree arc, and finally the semicircle and then the flipped-tip. Structure number (72) indicates the 45 degree curved, one centimeter long segment of the flipped-tip.

Distance from (55) to (56) is depth of distal semicircle, where distance of this depth is 0.75 cm. Distance between (57) and (58) is outer circumference of distal semicircle, and this distance is 1.5 cm. Region between position (57) and position (58) indicates distal-most region of catheter (FIG. 7), when the catheter is in relaxed position. But when the distal semicircle is flexed and caused to be a straight segment, then the distal-most structure of catheter takes the form of the flipped-tip.

Structure number (73) indicates 4 French outer diameter (OD) and also indicates 0.038 inner diameter (ID) of aperture that occurs at the very end of flipped-tip (FIG. 7).

Structure (61) is distal segment catheter length, which is 13.7 centimeters. Structure (62) is proximal segment catheter length, which is either 101.3 cm or 111.3 cm. Total length of catheter is either 13.7 cm plus 101.3 cm, which equals 115 em, or 13.7 cm plus 111.3 cm, which equals 125 cm.

Alternative Design of Catheter with Flipped-Tip (FIG. 7).

Alternative design of catheter with flipped-tip does not have the 2 two centimeter tapered straight region that is from 6 French down to 4 French, and does not have the two centimeter straight segment that resides immediately distal to the tapered straight region, but instead of having this tapered straight segment and this straight segment, has a six centimeter tapered straight segment, that is tapered from 6 French down to 4 French.

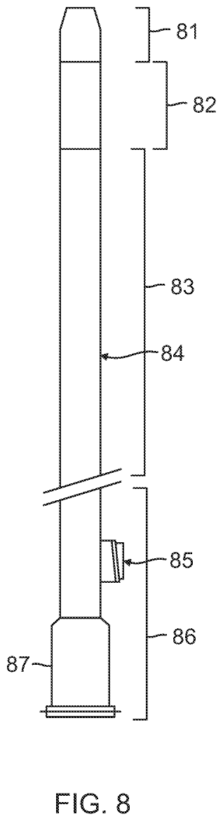

FIG. 8 shows balloon tip sheath. Structure (81) indicates sheath tip, where sheath tip is located about 2 cm from compliant balloon. Structure (82) indicates compliant balloon. Balloon is 3 centimeters long with inflated diameter of 7 millimeters maximal. Structure (83) is a shaft that is about 90-100 cm long. Structure (84) is internal balloon Inflation lumen. Structure (85) is proximal balloon inflation port. Structure (86) is port and hub assembly, which is about 10 centimeters in length. Structure (87) is hub.

Balloon exists on the outside of the guiding catheter or delivery sheath. There is a small parallel lumen adjacent to but completely separate from the main lumen, and which runs the entire length of the sheath/catheter. The balloon inflation lumen allows injection of liquid in the parallel port to inflate or deflate the balloon.

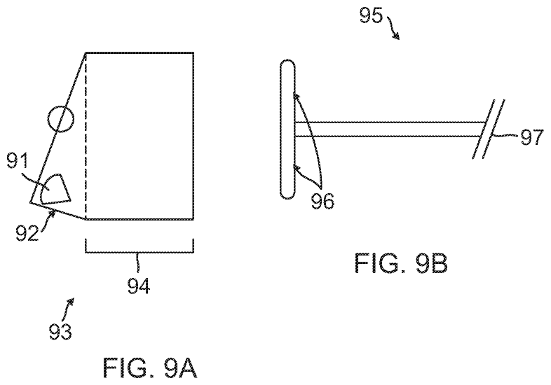

FIG. 9A and FIG. 9B show board base. Structure (91) is top view of board base. Structure (92) is spot for wrist pad. Structure (93) is attachment section to attach Velcro® adhesive pads. Structure (94) is fluoro table width. Structure (95) is edge-on view. Structure (96) is ledge, of about 15 millimeters, on both sides of board. Retains arm and is reversible. Structure (97) is 1 to 3 millimeters thick. Velcro® which is also known by the generic term, “velcro,” takes the form of a lawn of miniature hook-and-loop fasteners, where this law is attached to a fabric.

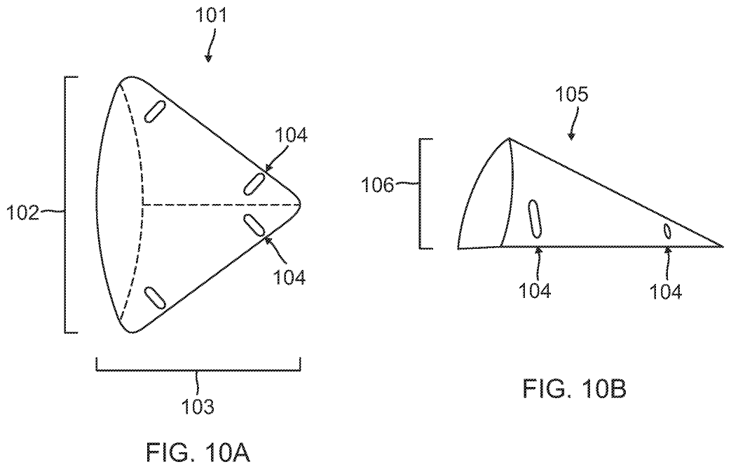

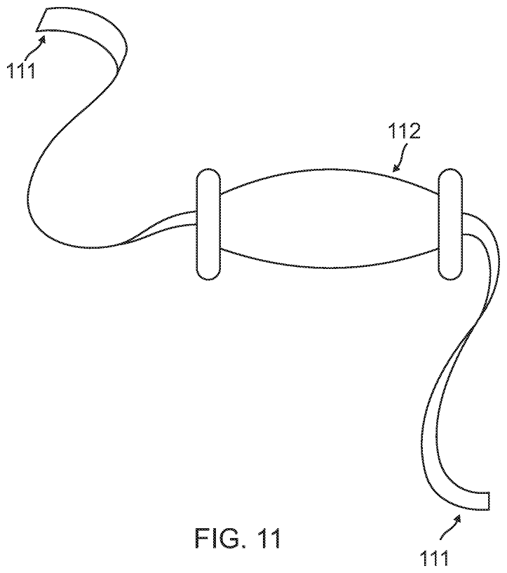

FIG. 10A and FIG. 10B show wrist pad. Structure (101) is top view of wrist pad. Structure (102) designates width of about 20 centimeters. Structure (103) designates width of about 20 centimeters. Structure (104) is strap holes for grip bar, where grip bar is shown in FIG. 11. Structure (105) is edge-on view of wrist pad. Structure (106) designates width of about 5 centimeters. Wrist pad has a shape that is somewhat an isosceles triangular shape that has an apex across which the wrist sits, causing the wrist to extend and expose the radial artery.

FIG. 11 shows grip bar. Structures (111) show Velcro ends that pass through strapholes of wrist band and fixate to Velcro on underside. Structure (112) is rubber or rubberized material.

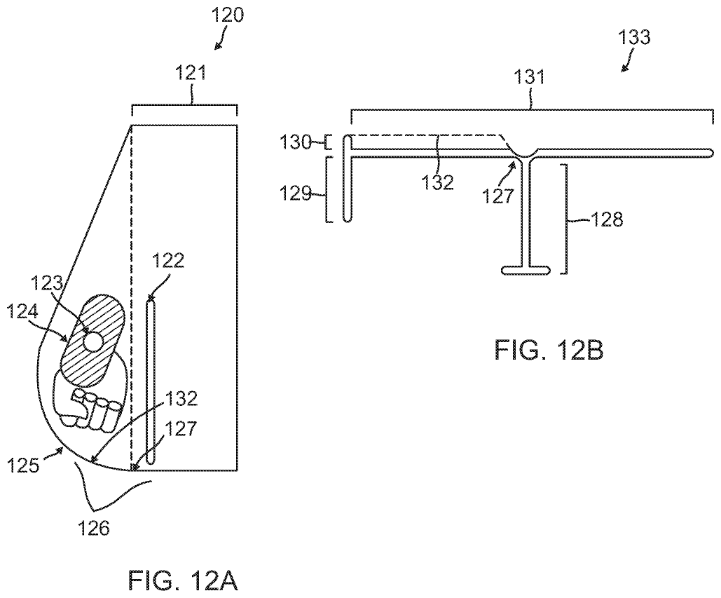

FIG. 12A and FIG. 12B show table top. Structure (120) is top view and structure (133) is side view. Structure (121) designates width of about 15 centimeters. Structure (122) is support stand see-through for illustration. Structure (123) is access site. Structure (124) is exposure cutaway. Structure (125) is 5 millimeter ledge that runs along outer edge, shown by dotted line. Structure (126) shows possible variations in overall length and shape of end segment. Structure (127) shows possible gutter to which drain bag attaches, where this structure number points to the TOP VIEW and also points to the EDGE-ON VIEW. Structure (128) is support stand, about 12 centimeters, optionally with adjustable mechanism. Structure (129) designates length of about 5 centimeters. Structure (130) designates length of about 5 millimeters. Structure (132) is curving ledge, where this structure number points to TOP VIEW and also to EDGE-ON VIEW. Possibly made of absorbent yet repellent material, like surgical drapes. Alternatively, can be sterilized and reused with custom shaped sterile drapes.

Table top secures the varying components of the positioning system while fixating everything to the fluoroscopy suite's tabletop. Table top ensures proper positioning of the components relative to one another.

Advantages of board base, wrist pad, grip bar, and table top. Board base, wrist pad, grip bar, and table top are all parts of a positioning system, wherein the grip bar looks like a handlegrip used with weight cables when lifting weights, where the grip bar is capable of being gripped by the patient when grip bar is attached to the board base, wherein the patient's wrist lies on the wrist pad, wherein the patient's wrist is in an extended position with artery fully exposed, and wherein the table top is an elevated component, and wherein the table top creates a flat surface between the arm and the torso of the patient, and wherein this flat surface is capable of mitigating the trench effect that occurs when the physician performs a transradial catheterization. Board base, table top, wrist pad, and grip bar are preferrably used together, that is, at the same time, but optionally can be used independently from one another. These components of the system of the present disclosure provide advantages when performing radial procedures, that is, radial insertion of catheter or radial insertion of assembled catheter, guidewire, and sheath.

CURVE EMBODIMENTS. This concerns segments of catheter of the present disclosure, where the segment defines a curve, and where the curve assumes a particular angle, or where the curve assumes a series of many angles, where the series takes the form of gradient of angles that increase in value, or where the series takes the form of a gradient of angles that decrease in value, or where the series takes the form of a gradient of angles that first increases in value and then decreases in value, or where the series takes the form of a gradient of angles that first decreases in value and then increases in value.

The above description relating to, “first increases in value and then decreases in value” or, “first decreases in value and then increases in value,” refers to the value of the angle (unit of degrees) as the physician, quality control analyst, or manufacturing technician, contemplates several progressing positions along the axis of a catheter, or contemplates several progressing positions along axis of a sheath. For a given segment of a catheter, the entire segment can define a curve an arc having a given angle, such as a twenty degree arc. Alternatively, a portion of that segment of the catheter, such as progressing positions along the axis of the catheter than correspond to the central 20% of a given curve, the central 30% of the curve, the central 40%, the central 50%, the central 60%, the central 70%, the central 80%, the central 90%, or the entire curve (where the entire curve of the curved segment equals 100% of that curve). FIG. 13 provides examples catheter segments that have a curve, and where the figure provides definitions of segments that correspond to the entire curve, or that correspond to the central 80% of the curve, and that correspond to the central 20% of the curve, and so on.

FIG. 13. Curved segments that share a common axis, where one or more of the segments can be used to define part of a cirle. The location where the vectors begin is at the very center of the circle. This figure can be used to define part or all of a curved region of catheter of the present invention. In various embodiments, a curved region of the catheter can include a curved region that is acquired from (carved out of) the image of FIG. 13. This acquired region can represent the entire curved region. Alternatively, this acquired region can be combined with zero, one, or more regions acquired from FIG. 14 (stretched area of oval) and with zero, one, or more regions acquired from FIG. 15 (squashed area of oval).

It is possible that most or perhaps all of the curved segments of cather of the present disclosure can be defined by segments of the semicircle of FIG. 13. For example, curves that appear to be almost flat can be defined as a ten degree slice from the curve of FIG. 13. But using FIG. 13 to define this almost flat curve to guide in manufacturing a catheter is almost impossible, because the user won't be able to compare this ten degree slice with a catheter that needs to be manufactured. To overcome this problem with perceiving, visualizing, and comparing, the curve of FIG. 14 can be used to define curves that are almost flat.

FIG. 14. Curved segments that share a common axis, where one or more of the segments can be used to define part of the stretched-out region of an oval (stretched, with respect to a circle). The location where the vectors begin was chosen, because it was a location where a variety of different shallow curves could be easily chosen, easily seen, and that readily provide guidance for manufacturing a curved segment of inventive catheter.

This figure can be used to define part or all of a curved region of catheter of the present invention. This defined region can be combined with zero, one, or more regions acquired from FIG. 13 and with zero, one, or more regions from FIG. 15.

FIG. 15. Curved segments that share a common axis, where one or more of the segments can be used to define part of the squashed region of an oval (squashed, with respect to a circle). This figure can be used to define part or all of a curved region of catheter of the present invention. This defined region can be combined with zero, one, or more regions from FIG. 13, and with zero, one, or more regions from FIG. 14.

HOW TO USE FIGS. 13,14, and 15 for defining catheter of the present disclosure, and for defining exclusionary embodiments. Where a catheter segment matches the conformation and curvature of a segment of a circle, it is sufficient that the description refer to, “a circle,” and here there is not any need to refer to FIG. 13. In contrast, where a catheter segment matches the conformation and curvature taken from part of FIG. 14 or FIG. 15, then it might be preferred to refer to FIG. 14 or FIG. 15, instead of referring to a circle.

FIG. 13 can be used for defining the shape of any curved region of any catheter providing the shape matches a segment of a circle. The entire circumference of a circle is 180 degrees. A tiny segment of a circle is almost a straight line, and tiny segments of a circle, for example, one degree or a half degree, can be used in descriptions to represent linear parts of a catheter. A typical curved segment in a catheter has a shape represented by a cut of the FIG. 13 circle, where the cut slices out a 45 degree pie slice.

SCALE. When FIG. 13, FIG. 14, and FIG. 15, are used in definitions of a catheter, what is used is only the shape and the relative length of the shape. But the actual lengths and the relative lengths of the circles and ovals in FIGS. 13, 14, and 15, are not used when defining an inventive circle. Each length of each shape (each shape taken from FIGS. 13, 14, and 15), is separately defined in inches, in centimeters, or in French units.

The location where the vectors begin was chosen, because it was a location where a variety of different shallow curves could be easily chosen, easily seen, and that readily provide guidance for manufacturing a curved segment of inventive catheter. Without implying any limitation on the present invention, the following discloses how to use the system of nomenclature that is provided by FIGS. 13, 14, and 15. Parts of the catheter are described, starting from proximal end of catheter (region that is held by physician) to distal end of catheter (region of catheter that is relatively close to patient's heart, during advancement of catheter via radial approach, or during advancement of catheter via femoral approach).

Catheter of FIG. 5.

Example Showing Use of FIG. 13 (Curved Segment from a Circle) to Describe Catheter of FIG. 5.

Catheter comprises a hub at the proximal terminus, a straight segment, wherein combined length of hub plus straight segment is 103.8 cm or 113.8 cm, a straight segment (3 centimeter long) that has a taper that gets narrower towards the distal direction (taper begins at 6 F and ends at 4 F), a straight segment (pre-taper segment) that has a length of 3 centimeters and a constant width of 4 F (outer diameter), followed by a 3 centimeter curved region that is curved to the left (curve segment) of about thirty degrees, and where this curve is followed by a semicircle (180 degrees) that defines the distal terminus of the catheter, and where this semicircle has an outer circumference diameter of 1.5 cm, and a radius of 0.75 cm, and where catheter has a total length as measured along outer surface, and where the distal tip defines an aperture with 4 French outer diameter and 0.038 inch inner diameter, and where the arc of the distal curve and the arc of the semicircle form a continuously curving arc that arcs only in the counterclockwise direction.

Alternate dimensions for semicircular segment that is actually ovoid, where ovoid curves are definable with respect to FIG. 14 or FIG. 15. Dimension of semicircle can be 0.5 centimeter radius with 1.0 centimeter side-to-side diameter, or 0.5 centimeter radius with 1.5 centimeter side-to-side diameter, or 0.75 centimeter radius with 1.5 centimeter side-to-side diameter.

In “about” embodiments, dimension of semicircle can be about 0.5 centimeter radius with about 1.0 centimeter side-to-side diameter, or about 0.5 centimeter radius with about 1.5 centimeter side-to-side diameter, or about 0.75 centimeter radius with about 1.5 centimeter side-to-side diameter. The above dimensions, and alternate dimensions, can be applied to catheter of FIG. 5, of FIG. 6 (has S-curve), or of FIG. 7 (has distal terminal flipped-tip).

Catheter of FIG. 6.

Example Showing Use of FIG. 13 (Curved Segment from a Circle) to Describe Catheter of FIG. 6.

Catheter of FIG. 6 comprises a hub at the proximal terminus, a straight catheter segment, wherein length of hub plus straight catheter is 103.8 or 113.8 centimeters long, a tapered straight segment (3 centimeter long) that has a taper that gets narrower towards the distal direction (taper begins at 6 F and ends at 4 F), an S-curve that consists of a first curved segment and a second curved segment, where in the first curved segment that curves to the right, wherein the first curved segment is 3 centimeters long and where the curve assumes an arc of about 30 degrees, a second curved segment that curves to the left, where the second curved segment is 3 centimeters long and wherein the curve assumes an arc of about 30 degrees, and where this second curved segment is followed by a semicircle segment, and wherein the semicircle segment has an outside radius of about 0.5 centimeters, and an outside left-to-right diameter of between 1.0 cm and 1.5 cm, and where distal terminus comprises an aperture, wherein the aperture is defines the distal terminus of catheter tube, wherein aperture has an outer diameter of about 4 French (0.052 inches) and an inner diameter of about 0.038 inches.

1 French is equivalent to 0.33 millimeters. Also, 1 French is equivalent to 0.013 inches.

Catheter of FIG. 7.

Catheter embodiment of FIG. 7 has a distal terminal flipped-tip. Catheter of FIG. 7 comprises a hub at the proximal terminus, a straight catheter segment, wherein the combined length of hub plus straight catheter segment is 101.3 or 113.8 centimeters long, a straight segment (3 centimeter long) that has a taper that gets narrower towards the distal direction (taper begins at 6 F and ends at 4 F), a segment that is a 6 centimeter long curve taking the form of a 45 degree arc to the left, wherein the distal end of said 6 cm segment is followed by a semicircle that also arcs to the left, and wherein the semicircle has a 1.5 cm side-to-side diameter and a 0.75 radius, and wherein said semicircle is followed by a 1-centimeter long flip-out arc, and wherein the flip-out are terminates in an aperture, wherein the aperture is defined by distal terminus of catheter tube, wherein distal terminus of tube has an outer diameter of about 4 French and an inner diameter of about 0.038 inches, and wherein the 1 centimeter long flip-out arc has a curvature of 45 degrees.

Curvature Embodiments.

Catheter of the present disclosure can include one or more curved segments, where the curvature of the segment is preferably definable with reference to either FIG. 13 (figure of a circle). Alternatively, curvature can be defined with reference to FIG. 14 (region of an oval) or to FIG. 15 (region of an oval).

Segment can assume a curvature of 2 degrees, 4 degrees, 6 degrees, 8 degrees, 10 degrees, 15 degrees, 20 degrees, 25 degrees, 30 degrees, 35 degrees, 40 degrees, 45 degrees, 50 degrees, and so on. Also, segment can assume a curvature of about 2 degrees, about 4 degrees, about 6 degrees, about 8 degrees, about 10 degrees, about 15 degrees, about 20 degrees, about 25 degrees, about 30 degrees, about 35 degrees, about 40 degrees, about 45 degrees, about 50 degrees, and so on, where the term “about” can mean, plus or minus 0.5 degree, plus or minus 1 degree, plus or minus 2 degrees, plus or minus 4 degrees, plus or minus 6 degrees, plus or minus 8 degrees, plus or minus 10 degrees, and so on.

Also, segment can assume a curvature of 2-5 degrees, 3-6 degrees, 4-7 degrees, 5-8 degrees, 6-9 degrees, 7-10 degrees, 8-11 degrees, 9-12 degrees, 10-13 degrees, 11-14 degrees, 12-18 degrees, 15-21 degrees, 18-24 degrees, 21-27 degrees, 24-30 degrees, 27-33 degrees, 30-36 degrees, 33-39 degrees, 36-42 degrees, 39-45 degrees, 42-48 degrees, 45-51 degrees, 48-54 degrees, 51-57 degrees, and so on. In exclusionary embodiments, system, medical device, catheter, and methods of the present disclosure can exclude any system, medical device, or catheter that comprises a segment definable by one of the above curvatures or by one of the above curvature ranges.

Length Embodiments.

A segment of catheter of the present disclosure can take the form of, for example, a hub, a straight segment that includes a hub, a straight segment that does not include a hub, a tapered segment, a straight segment, a curved segment that is not tapered, a curved segment that is tapered, and so on. For any given catheter that comprises one or more segments, lengths of each segment can be chosen from 0.5 centimeters (length of semicircle in FIG. 5 that has a 0.75 cm radius), 3 cm (e.g., length of distal curve in FIG. 5), 6 cm (e.g.; taper of FIG. 5), 103.8 cm or 113.8 cm (e.g., distal catheter of FIG. 5).

S-SHAPED CURVE. FIG. 6 discloses a catheter with S-shaped curve that consists of two segments, proximal curved segment and distal curved segment. In a preferred embodiment, each segment has the same length, as measured through the central axis of the catheter lumen. In a preferred embodiment, each curved segment is three centimeters long, and has a thirty degree are. In alternate preferred embodiments, one or both segments are 2.0 cm long, 2.2 cm, 2.4 cm, 2.5 cm, 2.6 cm, 2.8 cm, 3.0 cm, 3.2 cm, 3.4 cm, 3.5 cm, 3.6 cm, 3.8 cm, or 4.0 cm long. In range embodiments, segment length has one of the above values, plus or minus 0.1 centimeters, plus or minus 0.2 centimeters, plus or minus 0.3 centimeters, and soon. In other range embodiments, segment arc assumes a 20 degree arc, a 25 degree arc, a 30 degree arc, a 35 degree arc, or a 40 degree arc, wherein applicable range embodiments can be plus or minus one degrees, plus or minus two degrees, plus or minus three degrees, or plus or minus four degrees.

In exclusionary embodiments, system, medical device, and catheter of the present invention can exclude a medical device or any catheter, that possesses a segment with one of the above length values, that possesses a segment with a length that is greater than one of the above-disclosed length values, or that possesses a segment with a length that is less than one of the above-disclosed length values. In other exclusionary embodiments, the present invention can exclude a medical device or any catheter that possesses a curved segment that assumes one of the above-disclosed curve values (units of degrees), that possesses a curved segment with a curve value that is greater than one of the above-disclosed curve values (units of degrees), or with a curve value that is lesser than one of the above-disclosed curve values (units of degrees).

SEMICIRCLE, WITH OR WITHOUT TERMINAL ARC. Catheter of each of FIG. 6, FIG. 7, and FIG. 8, disclose catheter with semicircular tube at distal end, where this tube is part of the catheter. Semicircular tube can possess a Flexural Modulus, as measurable, for example, by ASTM D790 or by ISO178. The Flexural Modulus of said semicircular tube can match one of the values, or can be within one of the ranges, as disclosed elsewhere in this document. The terminal arc can also possess a Flexural Modulus that can match one of the values, or can be within one of the ranges, as disclosed elsewhere in this document.

### Exclusionary Embodiments

In exclusionary embodiments, the system, medical devices, parts of the medical devices, methods of manufacture, methods of diagnosis, and methods of treatment of the present disclosure can exclude various devices, structures, and parts. What can be excluded can be any system, method of treatment, medical diagnosis, medical device, and method that includes one or more of, obturator, dilator, trocar, splittable sheath, hub, Seldinger technique, non-splitable sheath, tabs, wings, coupling section, bulb, cap, coupler, luer lock, valve, housing, stylet, guidewire, metal coil, lever, compressible sleeve, helical wire, ribbon wire, needle, flange, gasket, washer, Storz lock, and so on.

In exclusionary embodiments, the present disclosure can exclude any method that uses a blood thinning agent, or that uses an agent that is an anti-coagulant.

The indefinite articles “a” and “an,” as used herein in the specification and in the claims, unless clearly indicated to the contrary, should be understood to mean “at least one.”

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language mans that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

In embodiments, the term “about” can refer to a value where the unit is, for example, viscosity (e.g., centipoise), length (e.g., micrometers, millimeters, centimeters), hardness (e.g., as measured by durometer method and Shore hardness scale), or concentration in a liquid, fluid, or paste (e.g., grams per mL or millimoles per liter), and the like. The term “about,” as applied to a given value, can mean plus or minus 0.5%, plus or minus 1.0%, plus or minus 2%, plus or minus 4%, plus or minus 6%, plus or minus 8%, plus or minus 10%, plus or minus 15%, plus or minus 20%, plus or minus 25%, and so on. When a given value resides in a list of values, the term “about” can refer to a range of values that encompasses and is limited to that particular given value, the next greater value in that list, and the next lower in that list. Alternatively, when a given value resides in a list of values, the term “about” can refer to a range of values that encompasses and is limited to that particular given value, a value residing at the half-way point to the next greater value, and a value residing at the half-way point to the next lower value.

The terms “a,” “an,” “the” and similar referents used in the context of describing the invention (especially in the context of the following claims) are to be construed to cover both the singular and the plural, unless otherwise indicated herein or clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A catheter that is capable of radial passage through the cardiovascular system, and capable of passage to the lumen of the aortic arch, wherein the catheter comprises a proximal end and a distal end, and wherein the proximal end to the distal end of the catheter comprises a lumen, and wherein the catheter comprises:
(i) An optional hub that comprises the proximal end,
(ii) A first straight segment, that is not tapered,
(iii) A second straight segment that is tapered,
(iv) Optionally, a third straight segment, that is not tapered,
(v) A third straight segment, that is not tapered,
(vi) A first curved segment,
(vii) A second curved segment, and
(viii) Optionally, an S-shaped region consisting of a first curved segment that resides in this S-shaped region, and a second curved segment that resides in this S-shaped region, wherein the first curved segment provides the bottom half of an S-shape, and the second curved segment provides the top half of the S-shape, wherein the bottom half is relatively proximal and the top half is relatively distal.

2. The catheter of claim 1, wherein the catheter is exemplified by FIG. 5, wherein the catheter comprises a proximal end and a distal end, wherein the proximal end to the distal end the catheter comprises a lumen, wherein the catheter comprises:
(i) A hub that comprises the proximal end,
(ii) A first straight segment, that is not tapered,
(iii) A second straight segment, that is tapered,
(iv) A third straight segment, that is not tapered,
(v) A first curved segment, and
(vi) A second curved semicircular segment that comprises the distal end of said catheter,
wherein the first curved segment and the second curved segment assume a continuously curving arc, and wherein the first curved segment possesses a first central point, and the second curved segment posseses a second central point, and wherein the first and second central points are relatively close to each other,
wherein the hub defines an axis, and wherein each segment defines an axis,
wherein the combined length of the hub plus the first straight segment has a value that is selected from a value that is between 100 cm to 115 cm,
wherein the second straight segment that Is tapered is about three centimeters long and has a taper that gets narrower from the proximal direction to the distal direction, wherein taper begins at 6 French (outer diameter) and ends at 4 French (outer diameter),
wherein the third straight segment has a length of 3 centimeters and a constant width of 4 French (outer diameter),
wherein the first curved region has a constant width of 4 French, and a length of about 3 centimeters as measurable along central axis of first curved region, wherein the first curved region assumes a thirty degrees arc,
wherein the second curved segment is a semicircular (180 degrees) segment with constant width of 4 French, and wherein second curved segment comprises the distal terminus of the catheter, and wherein the semicircle has an outer circumference diameter of 1.5 cm, and a radius of 0.75 cm, and
wherein catheter has a total length measurable along central axis, wherein total catheter length is 125 cm or 125 cm, and wherein the distal tip defines an aperture with 4 French outer diameter and 0.038 inch inner diameter, and wherein the arc of the distal curve and the arc of the semicircle together form an arc that has a J-shaped conformation and does not have an S-shaped conformation.

3. The catheter of claim 1, wherein the catheter is exemplified by FIG. 6, wherein the catheter comprises a proximal end and a distal end, wherein the catheter comprises a lumen, and wherein the catheter comprises:
(i) A hub that is located at the proximal end,
(ii) A first straight segment, that is not tapered,
(iii) A second straight segment, that is tapered,
(iv) An S-shaped curve that comprises first curved segment and a second curved segment, wherein the S-shaped curve is capable of allowing for easier engagement of the arteries from a radial approach, specifically the right carotid and a bovine left carotid,
(v) A third curved segment that comprises an aperture open to the environment of use, wherein the third curved segment also comprises said distal end, wherein the third curved segment terminates in a distal tip that comprises an aperture that opens into environment of use, and
wherein the hub defines an axis and wherein each segment defines an axis, and
wherein said first curved segment and said second curved segment assume an S-shaped curve, and wherein said second curved segment and said third curved segment assume a continuously curving arc that assumes a continuous J-shaped curve,
wherein the combined length of the hub plus the first straight segment has a value that is selected from a value that is between 100 cm to 115 cm,
wherein the total catheter length is 115 cm or 125 cm,
wherein the straight tapered segment is three centimeters long as measurable by axis of lumen of straight tapered segment, wherein the straight tapered segment has a taper that gets narrower from the proximal direction to the distal direction, wherein taper begins at 6 French (outer diameter) and ends at 4 French (outer diameter),
wherein the first curved region has a constant width of 4 French, and is about three centimeters long as measurable along central axis of first curved region,
wherein the second curved region has constant width of 4 French and is about three centimeters long,
wherein the third curved segment is a semicircular (180 degrees) segment with constant width of 4 French, and wherein the third curved segment comprises the distal terminus of the catheter, and wherein the semicircle has an outer circumference diameter of 1.5 cm and a side-to-side radius of 0.75 cm, and a distal-to-proximal radius of 0.50 cm, and
wherein the catheter has a total length measurable along central axis, and wherein the distal tip possesses an aperture with 4 French outer diameter and 0.038 inch inner diameter, and wherein the aperture is open to the environment of use.

4. The catheter of claim 1, wherein the catheter is exemplified by FIG. 7, wherein the catheter comprises a proximal end and a distal end, wherein the catheter defines a lumen that extends from proximal end to distal end, wherein the catheter comprises:
(i) A hub that is located at proximal end end,
(ii) A first straight segment, that is not tapered,
(iii) A second straight segment, that is tapered,
(iv) A third straight segment, that is not tapered,
(v) A first relatiely long curved segment,
(vi) A second curved segment that assumes a semicircle,
(vii) A flipped-tip segment that comprises a 0.5 centimeter straight segment followed by a one centimeter long arc that assumes a 45 degree curve, wherein the flipped-tip segment comprises an aperture that is open to the environment of use,
wherein the first curved segment together with the second curved segment assume a J-shaped arc,
wherein the second curved segment together with the third curved segment assume an S-shaped arc,
wherein the hub defines an axis and wherein each segment defines an axis, and
wherein the combined length of the hub plus the first straight segment has a value that is selected from a length that is between 100 cm and 115 cm,
wherein the second straight segment, that is tapered, is two centimeters long as measurable along axis of lumen of straight tapered segment, wherein the second straight segment, that is tapered, has a taper that gets narrower when moving from the proximal to distal direction, wherein taper begins at 6 French (outer diameter) and ends at 4 French (outer diameter),
wherein the third straight segment, that is not tapered, has a width of 4 French and a length of 2 centimeters,
wherein the first curved segment has a constant width of 4 French, and has an arc of 45 degrees, and is about six centimeters long as measurable along central axis of first curved region,
wherein the second curved segment assumes a 180 degree semicircular arc, with constant width of 4 French, and
wherein the third curved segment comprises the distal terminus of the catheter, and wherein the semicircular arc has an outer circumference diameter of 1.5 cm, and a radius of 0.75 cm, and
wherein catheter has a total length measurable along central axis, wherein the total length is either 115 cm or 125 cm, and wherein the distl tip possesses a 4 French outer diameter and 0.038 inch inner diameter, and wherein the distal tip terminates in an aperture that is open to the environment of use.

5. The catheter of claim 1 that comprises a hub.

6. The catheter of claim 1 that does not comprise any hub.

7. The catheter of claim 1, in combination with a guidewire with a stiff “arch” segment that is about 150 centimeters long, followed in the proximal to distal direction with a tapered region that is about ten centimeters long, followed in the proximal to distal direction, with a tapered segment that is about twenty centimeters long where the taper is from about 0.035 inches down to about 0.012 inches, where the guidewire terminates with a microwire that is about 20 centimeters long, and where this microwire terminates with a tip with diameter of from 0.012 inches to 0.018 inches, and where optionally, a torque device is added onto the shaft of the guidewire at or near the guidewire's proximal end, and secured onto the proximal shaft.

8. A kit comprising the catheter of claim 1, wherein the kit further comprises one or more of a balloon tip sheath, a board base, a wrist pad, a grip bar, a table top,
wherein the balloon of the balloon tip sheath exists on the outside of the sheath, and wherein there is a small parallel lumen adjacent to but completely separate from the main lumen, and which runs the entire length of the sheath, and wherein the sheath comprises a parallel port, and wherein balloon inflation lumen allows injection of liquid in the parallel port to inflate the balloon and also allows removal of liquid from the parallel port to deflate the balloon,
and wherein board base, wrist pad, grip bar, and table top are all parts of a positioning system, wherein the grip bar looks like a handlegrip used with weight cables when lifting weights, where the grip bar is capable of being gripped by the patient when grip bar is attached to the board base, wherein the patient's wrist lies on the wrist pad, wherein the patient's wrist is in an extended position with artery fully exposed, and wherein the table top is an elevated component, and wherein the table top creates a flat surface between the arm and the torso of the patient, and wherein this flat surface is capable of mitigating the trench effect that occurs when the physician performs a transradial catheterization.

9. An alternate design catheter, as defined herein, where in the alternate design catheter comprises a tapered straight region that is about six centimeters long, and that does not include any tapered straight region that is between about two centimeters long and about three centimeters long, wherein the alternate design catheter is:
(i) The alternative design of the basic catheter,
(ii) The alternative design of the catheter with an S-shaped curve, or
(iiii) The alternative design of the catheter with the flipped-tip,

10. A method for manufacturing the catheter of claim 1, wherein the method uses a mandrel, wherein the mandrel comprises polytetrafluoroethylene (PTFE) liner coating, and wherein the mandrel is a scaffold for manufacturing said catheter, the method comprises the steps of:
(a) The step of cutting the mandrel to the desired size,
(b) The step of placing the mandrel in a machine that comprises spools of stainless steel and nitinol,
(c) The step where the machine applies the stainless steel and nitinol coil winds on top of the PTFE liner on the mandrel, in order to form a pattern that is a single wire of stainless steel followed by three wires of nitinol with varying thicknesses, wherein the catheter excludes any coil winding at the distal 1.0 to 1.5 centimeters,
(d) The step of repeating the nitinol-stainless steel pattern on the proximal shaft (shaft closest to hub),
(e) The step of applying a coil consisting of only nitinol and without any steel, on the distal shaft,
(f) The step wherein optionally, nitinol and stainless steel are joined together by laser welding,
(g) The step wherein polymers are applied after the metal is applied, wherein polymer sections of varying stiffnesses according to the desired flexibility of the catheter section are placed over the metal,
(h) The step wherein said polymers are then bonded to the catheter section underneath it with heat treating, optionally and preferrably where it is suspended top to bottom to allow the heat set to bind the coil to the polymer,
(i) The step where a hydrophilic coating is applied to the distal section, via dipping with a mandrel inside, usually on the distal aspect, but typically not at the segment that interacts with the arch, approximately 30 mm section, halfway along the catheter, wherein for a preferred design dip the shaft to coat it with the antivasospasm agent,
(j) The step wherein optionally, a hub is affixed to proximal end of the catheter,
(k) The step wherein, optionally, anti-vasospasm drug in an excipient is applied to the catheter as a coating, wherein excipient can comprise a hydrogel or a time-release formulation or a hydrophilic polymer, and wherein the sum of all segments of catheter comprises catheter shaft, and wherein the coating is applied to entire catheter shaft, or to entire catheter shaft but not to semicircular segment, or to entire catheter shaft but not to flipped-tip and not to semicircular segment,
(l) Wherein said method produces a final catheter has three layers from hub to tip, wherein from inside to outside, there is a polytetrafluoroethylene (PTFE) liner section, a metal coil wind section, and a polymer section Typically a hub can be affixed, and the hub is optional.

11. The catheter of claim 1, that comprises a coating, wherein the coating comprises an anti-vasospasm drug, and wherein the anti-vasospasm drug is capable of release in an amount sufficient to reduce the frequency or intensity of spasms of blood vessels when said catheter is inserted into a patients vasculator and then passed through the patients vasculature towards the aortic arch, or when at least part of the catheter is inserted into and the patient's aortic arch.

12. The catheter of claim 1 that is manufactured by the method of claim 10.

13. The catheter of claim 1, wherein the catheter comprises a supply of an anti-vasospasm drug, and wherein the catheter is capable of releasing the anti-vasospasm drug from the sheath's sidearm through small channels or through rivulets or through laser-cut holes from the catheter that run along the shaft of the catheter, wherein the channels, rivulets, or holes do not allow blood to enter the sheath.

14. A sheath that comprises a shaft, wherein the sheath is capable of being used to introduce the catheter of claim 1 into the vasculature of a patient, wherein the sheath comprises a supply of an anti-vasospasm drug, and wherein the sheath comprises a sidearm, wherein the sheath is capable of releasing the anti-vasospasm drug from the sheath's sidearm through small channels or through rivulets or through laser-cut holes from the sheath sidearm that run along the shaft of the sheath, wherein the channels, rivulets, or holes do not allow blood to enter the sheath.

15. A method for using the catheter of claim 1 in a patient, for the treatment or diagnosis of a cardiovascular condition in the patient, the method comprising one or more or all of the steps of:
(i) The step of inserting a guidewire into the catheter to form an assembled catheter plus guidewire, followed by inserting the assembled catheter plus guidewire into a sheath, to form an assembled guidewire plus catheter plus sheath,
(ii) The step of inserting the catheter into a sheath, to form an assembled catheter plus sheath, followed by inserting a guidewire into the catheter, to form an assembled guidewire plus catheter plus sheath,
(iii) The step of inserting the assembled guidewire plus catheter plus sheath into the patient's vasculature wherein the inserting is at the radial vasculature or at the femoral vasculature,
(iv) The step of pushing at least the catheter of the assembled guidewire plus catheter plus sheath into the patient's aortic arch,
(v) The step of straightening out the flipped-tip that resides at the distal end of the catheter, wherein the a physician accomplished the straightening by using the physician's own hands, in the situation where the catheter possesses a flipped-tip at the distal end, and wherein the step of straightening is optionally performed at the time that catheter is inserted into a sheath, or at the time that assembled guidewire plus catheter is inserted into a sheath.

16. A system for transradial access of the vasculature, which comprises, in combination:
a plurality of specialty shaped and formed catheters;
a grouping of compliant balloons, sheaths and wire tools; and
wherein said catheter lengths are ranging between at least about 111 and 127 centimeters;
having approximately 3.5 to 5.5 French diameters throughout.

17. The system of claim 16, and the entire disclosure, further comprising:
at least a curved inner catheter optimized for selection of the origins of the arteries in the body.

18. The system of claims 16 and 17, and the entire disclosure, further comprising:
The plurality of specialty shaped catheters being coaxially introduced inside of another catheter which is lubricious and having compliance in certain segments for advancement.

19. The system of claim 18, wherein each of the plurality of catheters are introduced simultaneously or in alternating fashion to advance into a select artery after initial engagement.

20. The system of claims 16-19, and the entire disclosure for providing a means for accessing the endoluminal cerebral vasculature from the radial artery.

21. A novel enhanced system for transradial cerebral access, as shown and described herein, and different from known systems, comprising in combination, at least a kit,
further comprising:
at least three sets of specialty catheters;
a wire, sheath and introducer in predetermined size ranges, and methods for optimizing combination of catheters and said other tools.

22. A Modular Positioning System (MPS), as shown and described, further comprising, in combination:
a board base;
a grip bar; and
a wrist pad.

23. The modular positioning system (MPS) of claim 22, the figures and entire disclosure as shown and described is a table-top version.

24. The MPS of claim 22, as disclosed further comprising absorbent yet hydrophobic/repellent disposable material.

25. The MPS of claim 22, shown and described, further comprising:
wrist pad locators, with attachment sections;
ledges; and a plurality of apertures.

26. The MPS of claim 22, shown and described for facilitating radial access.

27. The MPS of claim 22, shown and described for general vascular access.

28. The MPS of claim 22, shown and described for transradial cerebral access.

29. A kit, comprising transradial access tools including a set of curve specific catheters, introducers and specialized wires.

30. The kit of claim 29, and the disclosure further comprising an MPS.

31. The kit of claims 29 and 30, that is capable of transradial cerebral access.

32. A transradial access system, comprising, in combination:
a non-transfemoral approach to neuro-endovascular procedures comprised of catheter assemblies ranging from at least about 111 to 127 cm's, with Fr sizes between at least about 3.2 to 5.9; a plurality of specialized curves emplaced within said catheter assemblies; specialized wires, sheaths and introducers.

33. The system of claim 32 that is capable of cerebral access.

34. The system of claim 33 that is capable of geriatric use being further specialized, customized and adapted to challenging vessel morphology.
